(19) United States
(12) Patent Application Publication
(10) Pub. No.: US 2017/0224823 A1
(43) Pub. Date: Aug. 10, 2017

(54) PLATINUM-CATALYZED SILICONE DRUG DELIVERY DEVICES AND METHODS OF USE THEREOF

(71) Applicant: International Partnership For Microbicides, Inc., Silver Spring, MD (US)

(72) Inventors: Wendy Blanda, Tustin, CA (US); Jonathon Daryll Holt, Durham, NC (US); Andrew Nathan Brimer, Bethlehem, PA (US); Karl Malcolm, Belfast (GB); Clare McCoy, Newtonabbey (GB); Diarmaid Murphy, Belfast (GB); Peter John James Boyd, Ballyclare (GB)

(21) Appl. No.: 15/519,280

(22) PCT Filed: Oct. 22, 2015
(86) PCT No.: PCT/US15/56814

§ 371 Date: Apr. 14, 2017

Related U.S. Application Data
(60) Provisional application No. 62/067,122, filed on Oct. 22, 2014.

(51) Int. Cl. A61K 47/02; A61K 31/567; A61K 31/505; A61K 9/00

(52) U.S. Cl. CPC A61K 47/02; A61K 9/0036; A61K 31/567; A61K 31/505

(57) ABSTRACT

The present invention provides intravaginal drug delivery devices, such as intravaginal rings, comprising active pharmaceutical ingredients (APIs) having terminal alkene, alkyne or carbonyl functionalities. The devices of the invention exhibit increased recovery of the active pharmaceutical ingredient from platinum-catalyzed silicone polymers due to the optimization of drug particle size and cure conditions. The present invention also provides methods of preventing unintended pregnancy in a female human, methods of preventing unintended pregnancy in a female human and HIV infection in a female human, and methods of preparing intravaginal drug delivery devices.

### RELATED APPLICATION INFORMATION

This application claims priority to U.S. Provisional Application No. 62/067,122, filed on Oct. 22, 2014, the entire contents of which are expressly incorporated herein by reference.

### BACKGROUND OF THE INVENTION

Silicone elastomer materials are employed in a wide range of applications, including medical devices and drug delivery devices. In drug delivery applications, it is often desirable for the silicone elastomer material to effectively control the release of one or more active pharmaceutical ingredients (APIs) over extended time periods, in order to achieve a prolonged duration of therapeutic effect and thereby improve clinical outcomes. Typically, the one or more active pharmaceutical ingredient is added to at least one component of the silicone elastomer system, the components of which are subsequently mixed and cured under elevated temperature conditions to form the final drug delivery product. In order to facilitate release of the API(s) from the silicone elastomer drug delivery system, the API(s) must possess some measure of solubility in the cured silicone elastomer material, and the solubilized molecules must also be capable of diffusing through the cured silicone elastomer material. Any constraint placed upon these solubility and/or diffusional processes by the silicone elastomer material may lead to reduced release or complete absence of release.

Silicone elastomer systems having different cure chemistries are well known in the art. For medical device and drug delivery applications, ‘condensation-cure’ and ‘addition-cure’ silicone elastomer systems are used most commonly. Tin-based compounds are often used to catalyze the former, while platinum-based compounds are used as a catalyst for the latter. It is also well known in the art that certain chemical substances, and more particularly, certain chemical functional groups, can interfere with or inhibit the addition-cure hydrosilylation reaction. For example, certain compounds containing sulfur (e.g., sulfides, thio compounds, etc.), nitrogen (amides, amines, nitriles, etc.), tin or phosphorous are known to inhibit the addition-cure reaction by poisoning the platinum-containing catalyst. Moreover, for APIs containing certain chemical functional groups capable of undergoing a hydrosilylation reaction, there is the possibility of the API undergoing an addition-cure reaction with key components of the silicone elastomer material, such that the API is covalently and permanently bound to the silicone elastomer material and thus no longer available for release from the device. Surprisingly, despite a long history of silicone elastomer materials incorporating APIs containing functional groups capable of undergoing hydrosilylation reaction, the chemical binding of such APIs with silicone elastomer materials has not been previously reported, much less a solution to the problem offered.

Specifically, no formulations have been discovered whereby active pharmaceutical ingredients (APIs) having alkene, alkyne or carbonyl functionalities have been incorporated into a platinum-cured silicone intravaginal rings or other drug delivery devices with API recovery in the 75-100% range due to covalent binding. Accordingly, there remains a need for the development of improved platinum-catalyzed silicone drug delivery devices with high API recovery rates. Specifically, there remains a need for improved platinum-catalyzed silicone intravaginal rings comprising a contraceptive to prevent unintended pregnancy, as well as intravaginal rings comprising a contraceptive agent in combination with an antimicrobial agent to prevent unintended pregnancy and HIV transmission.

### SUMMARY OF THE INVENTION

The present invention encompasses methods for reducing the binding of suitably-functionalized active pharmaceutical ingredients when incorporated into addition-cure silicone elastomer materials, such that an acceptable fraction of the active pharmaceutical ingredient is present and quantifiable in a physical state suitable for release from the drug delivery device, and the release of the active pharmaceutical ingredient(s) from the silicone elastomer material is optimized. Specifically, the present invention provides drug delivery devices, such as intravaginal rings, comprising active pharmaceutical ingredients (APIs) having terminal alkene, alkyne or carbonyl functionalities, which exhibit increased recovery from platinum-catalyzed silicone polymers due to the optimization of drug particle size and cure conditions. Only by decreasing the amount of drug solubilized in the silicone during the cure cycle will the amount of drug bound to the silicone polymer be reduced to an acceptable level for commercial drug products. Specifically, the instant inventors have discovered that the amount of drug which is solubilized after mixing into the silicone system is determined by three factors: 1) total surface area of the drug exposed to the silicone polymer; 2) the temperature of the system, and 3) the time the drug has been in contact with the silicone. Thus, drug solubilization can be reduced by using highly crystalline drug material (so that more energy is needed to solubilize the drug), by using larger crystal size, which reduces the surface area to increase solubilization time, and by reducing thermal exposure. By optimizing intravaginal ring formulations for drug particle size, cure temperature and cure time, the instant invention minimizes the amount of drug which binds to a silicone polymer during the platinum curing process, thereby resulting in higher recovery of the drug from platinum-catalyzed silicone intravaginal drug delivery devices.

In one aspect, the invention provides a platinum-catalyzed silicone intravaginal drug delivery device comprising a compound having a terminal alkene, a terminal alkyne, or a terminal carbonyl, wherein at least about 75% of the compound is recoverable from the device. In one embodiment, at least about 80% of the compound is recoverable from the device. In another embodiment, at least about 85% of the compound is recoverable from the device. In another embodiment, at least about 90% of the compound is recoverable from the device. In another embodiment, at least about 95% of the compound is recoverable from the device. In yet another embodiment, at least about 99% of the compound is recoverable from the device.

In another aspect, the invention provides a platinum-catalyzed silicone intravaginal drug delivery device comprising a compound having a terminal alkene, a terminal alkyne, or a terminal carbonyl, wherein about 100% of the compound is recoverable from the device.

In yet another aspect, the invention provides a platinum-catalyzed silicone intravaginal drug delivery device comprising a compound having a terminal alkene, a terminal alkyne, or a terminal carbonyl, wherein less than about 25% of the compound is covalently bound to the silicone. In one embodiment, less than about 20% of the compound is covalently bound to the silicone. In one embodiment, less than about 15% of the compound is covalently bound to the silicone. In another embodiment, less than about 10% of the compound is covalently bound to the silicone. In another embodiment, less than about 5% of the compound is covalently bound to the silicone. In another embodiment, less than about 2% of the compound is covalently bound to the silicone. In yet another embodiment, less than about 1% of the compound is covalently bound to the silicone.

In another aspect, the invention provides a platinum-catalyzed silicone intravaginal drug delivery device comprising a compound having a terminal alkene, a terminal alkyne, or a terminal carbonyl, wherein about 0% of the compound is covalently bound to the silicone.

In one embodiment of the invention, the compound is covalently bound to a silicone hydride group on the silicone polymer. In another embodiment, the compound is present in the device in a therapeutically effective amount. In another embodiment, the compound is present in the device in a prophylactically effective amount.

In another embodiment, the compound has a large primary particle size. In one embodiment, the compound has a d50 of about 40 microns to about 500 microns. In another embodiment, the compound has a d50 of about 40 microns to about 250 microns. In yet another embodiment, the compound has a d50 of about 55 microns to about 100 microns. In another embodiment, the compound has a d50 of about 55 microns.

In another embodiment, the compound has a d90 of about 80 microns to about 500 microns. In another embodiment, the compound has a d90 of about 80 microns to about 250 microns. In yet another embodiment, the compound has a d90 of about 80 microns to about 150 microns. In another embodiment, the compound has a d90 of about 135 microns. In a further embodiment, the compound has a primary particle size of about 40 microns to about 500 microns.

In one embodiment, the compound is non-micronized.

In another embodiment, the silicone is NuSil MED-4870 or NuSil DDU-4320.

In a further embodiment, the compound is a contraceptive. In one embodiment, the contraceptive is selected from the group consisting of levonorgestrel (LNG), ethynyl estradiol, noresthisterone, ethynodiol diacetate, desogestrel, and lynestrenol. In one embodiment, the contraceptive is levonorgestrel. In one embodiment, about 16 mg to about 64 mg of the contraceptive is present in the intravaginal drug delivery device. In another embodiment, about 32 mg of the contraceptive is present in the intravaginal drug delivery device.

In another embodiment, the intravaginal drug delivery device further comprises an antimicrobial compound. In one embodiment, the antimicrobial compound is dapivirine. In one embodiment, about 25 mg to about 400 mg of dapivirine is present in the intravaginal drug delivery device. In another embodiment, about 200 mg of dapivirine is present in the intravaginal drug delivery device.

In one embodiment, the intravaginal drug delivery device is a matrix-type intravaginal ring. In another embodiment, the intravaginal drug delivery device is a reservoir-type intravaginal ring.

In another aspect, the invention provides methods for producing a platinum-catalyzed silicone intravaginal drug delivery device comprising a compound having a terminal alkene, a terminal alkyne or a terminal carbonyl group, wherein at least 75% of the compound is recoverable from the device, the method comprising a) preparing a premix comprising the compound, b) transferring the premix to an injection molder, and c) curing the premix for about 60 seconds to about 10 minutes at a temperature of about 60° C. to about 120° C., wherein the compound has a primary particle size of about 40 microns to about 500 microns, thereby producing the platinum-catalyzed silicone intravaginal drug delivery device.

In one embodiment, the premix is cured for about 60 seconds, about 90 seconds, about 3 minutes or about 10 minutes. In another embodiment, the premix is cured at a temperature of about 60° C., about 80° C., about 100° C., or about 120° C. In one embodiment, the compound has a d90 of about 80, about 90, about 100, about 110, about 120, about 130, about 135, about 140, about 160, about 180, or about 200.

In another aspect, the invention provides methods of preventing pregnancy in a female human, comprising the step of inserting the intravaginal drug delivery device of the invention into the vagina of the female human.

In another embodiment, the invention provides methods of preventing pregnancy and preventing HIV infection in a female human, comprising the step of inserting the intravaginal drug delivery device of the invention into the vagina of the female human.

In one aspect, the present invention provides a method for incorporating substances possessing one or more unsaturated chemically-reactive functional groups, including active pharmaceutical ingredients (APIs), into addition-cure silicone elastomer materials such that any chemical reaction that might occur between the substance(s) and the silicone elastomer material is minimized and the substance is substantially available for release from the silicone elastomer material.

In another aspect, the invention provides a method for incorporating substances possessing one or more unsaturated chemically-reactive functional groups, including active pharmaceutical ingredients (APIs), into intravaginal ring devices, said intravaginal rings fabricated from addition-cure silicone elastomer(s), such that any chemical reaction that might occur between the substance(s) and the silicone elastomer material is minimized and the substance is substantially available for release from the intravaginal ring device.

In another aspect, the invention provides a method for incorporating steroid substances possessing one or more unsaturated chemically-reactive functional groups, into intravaginal ring devices fabricated from addition-cure silicone elastomer(s), such that any chemical reaction that might occur between the steroid substance and the silicone elastomer material is minimized and the substance is substantially available for release from the intravaginal ring device.

In another aspect, the invention provides a method for incorporating levonorgestrel into intravaginal ring devices fabricated from addition-cure silicone elastomer(s), such that any chemical reaction that might occur between levonorgestrel and the silicone elastomer material is minimized and the substance is freely available for release from the intravaginal ring device.

In yet another aspect, the invention provides a method for incorporating levonorgestrel into intravaginal ring devices fabricated from addition-cure silicone elastomer(s) under low cure temperature conditions, such that any chemical reaction that might occur between levonorgestrel and the silicone elastomer material is minimized and the substance is substantially available for release from the intravaginal ring device.

In yet another aspect, the invention provides a method for incorporating levonorgestrel into intravaginal ring devices fabricated from addition-cure silicone elastomer(s) under short cure time conditions, such that any chemical reaction that might occur between levonorgestrel and the silicone elastomer material is minimized and the substance is substantially available for release from the intravaginal ring device.

In yet another aspect, the invention provides a method for incorporating levonorgestrel having non-micronized particle size into intravaginal ring devices fabricated from addition-cure silicone elastomer(s), such that any chemical reaction that might occur between levonorgestrel and the silicone elastomer material is minimized and the levonorgestrel is substantially available for release from the intravaginal ring device.

In yet another aspect, the invention provides a method for incorporating levonorgestrel having non-micronized particle size into intravaginal ring devices fabricated from addition-cure silicone elastomer(s) under low cure temperature and short cure time conditions, such that any chemical reaction that might occur between levonorgestrel and the silicone elastomer material is minimized and the substance is freely available for release from the intravaginal ring device.

Other features and advantages of the invention will be apparent from the following detailed description and claims.

### BRIEF DESCRIPTION OF THE DRAWINGS

FIG. 1 depicts the mean and cumulative release of dapivirine plotted against time for platinum catalyzed silicone rings stored under various storage conditions for two weeks. Error bars denote standard deviation. P: packaged, UP: unpackaged, RH: relative humidity.

FIG. 2 depicts the mean and cumulative release of levonorgestrel plotted against time for platinum catalyzed silicone rings stored under various storage conditions for two weeks. Error bars denote standard deviation. P: packaged, UP: unpackaged, RH: relative humidity.

FIG. 3 depicts the recovery of levonorgestrel (LNG) from silicone platinum catalyzed

slabs cured at 100° C. for 90 seconds by the source of LNG. Error bars denote standard deviation. NM: non-micronized LNG; d90: the diameter at which 90% of the sample's measured particles are smaller sized particles.

FIG. 4 depicts a microscopy image of Chemo LNG Batch No. C1375, which has a d90 of 294 μm. Although described by the supplier has having large particle size distribution, the micrographs clearly show that the bulk properties of C1375 is similar to those of micronized LNG and contain a combination of small primary particles and larger physically agglomerated particles. These agglomerates most likely account for the larger particle dimensions quoted by the material supplier.

FIG. 5 depicts a microscopy image of Chemo LNG Batch No. C1401, which has a d90 of 384 μm. Although described by the supplier has having large particle size distribution, the micrographs clearly show that the bulk properties of C1375 is similar to those of micronized LNG and contain a combination of small primary particles and larger physically agglomerated particles. These agglomerates most likely account for the larger particle dimensions quoted by the material supplier.

FIG. 6 depicts representative micrographs of Tecoland LNG_{NM} APIs as received. (a) Batch No. 120101, (b) Batch No. 02001201016, and (c) Batch No. 02001201017. All images were recorded at 200×.

FIG. 7 depicts representative micrographs of Chemo LNG_{C1401} API recorded at (a) 100× and (b) 300×.

FIG. 8(A) depicts primary particle size versus measured particle size. FIG. 8(B) depicts particle size distribution versus crystal size.

FIG. 9 depicts the influence of (a) cure time and (b) cure temperature on the recovery of non-micronized and micronized levonorgestrel (LNG) from silicone slabs prepared using NuSil DDU-4320 silicone.

FIG. 10 depicts the influence of (a) cure time and (b) cure temperature on the recovery of non-micronized and micronized levonorgestrel (LNG) from silicone slabs prepared using NuSil MED-4870 silicone.

FIG. 11 depicts the maximum recoverable levonorgestrel (LNG) content for non-micronized LNG in NuSil DDU-4320 platinum catalyzed silicone slabs or intravaginal rings.

FIG. 12 depicts Raman microscopy studies to assess drug binding to poly(methylhydrosiloxane). LNG: levonorgestrel, DAP: dapivirine, PRG: progesterone, EE: Ethynyl estradiol.

FIG. 13(A) depicts Karlstedt's catalyst interacting with terminal double bonds. FIG. 13(B) depicts levonorgestrel (LNG) binding to the silicone elastomer at both the alkyne and/or the carbonyl (α,β-unsaturated ketone) groups.

FIG. 14 depicts platinum-cured silicone chemistry.

FIG. 15 depicts contraceptive compounds which comprise a terminal double or triple bond: (a) Levonorgestrel (LNG), (b) Ethynyl Estradiol (EE), (c) Norethisterone, (d) Ethynodiol Diacetate, (e) Desogestrel, and (f) Lynestrenol.

### DETAILED DESCRIPTION OF THE INVENTION

Manufacturing of silicone drug delivery devices, e.g., intravaginal rings, comprising active pharmaceutical ingredients having a terminal alkene, alkyne or carbonyl group, such as steroidal hormones, e.g., levonorgestrel (LNG), involves the process of curing silicone polymers in the presence of the active pharmaceutical ingredient. Silicone curing transforms the silicone polymers into a three-dimensional network to produce an elastomer, and involves cross-linking, or formation of chemical bonds between adjacent silicone polymers. Several types of cross-linking reactions may be employed for silicone curing, such as cross-linking with radicals, cross-linking by condensation or cross-linking by addition. The latter reaction is a hydrosilylation reaction, carried out by reacting vinyl endblocked or vinyl branched polymers with Si—H groups carried by functional oligomers, as is shown below:

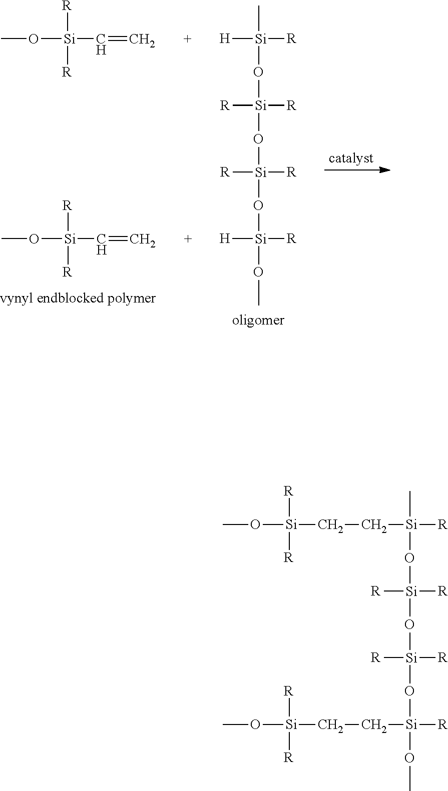

Cross-linking by addition involves the use of a catalyst, such as a platinum (Pt) or a ruthenium (Rh) catalyst. One example of the platinum catalyst is Karlstedt's catalyst having the structure as shown below:

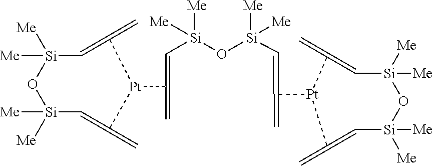

During the reaction, the catalyst interacts with the vinyl groups of the silicone polymer. Without wishing to be bound by a specific theory, it is believed that other compounds comprising alkene, alkyne or carbonyl functionalities, e.g., compounds having terminal double or triple bonds or carbonyl bonds, when present during silicone curing, may interact with the catalyst and substitute for the vinyl functional groups of the silicone backbone. This interaction results in the compound becoming covalently and irreversibly bound to the silicone polymer. Accordingly, it is believed that certain active ingredients comprising terminal double or triple bonds, or one or more carbonyl bond, may become irreversibly bound to the silicone polymer during the curing process. Such active ingredients that are irreversibly bound to the silicone polymer will not be bioavailable and will not elute from a nonbioerodible polymer network.

Any active pharmaceutical ingredient comprising double, triple or carbonyl bonds may covalently bind to the silicone polymer during the silicone curing process. Such active ingredient may be a steroid hormone, such as a contraceptive, that comprises a terminal alkyne, such as levonorgestrel, ethynyl estradiol, norethisterone, ethynodiol diacetate, desogestrel or lynestrenol. Other exemplary steroid hormones are the hormones that comprise one or more carbonyl (C═O) bonds, such as progesterone.

Structures of exemplary steroid hormones are shown in the table below.

| Name of Steroid | |
|---|---|
| Hormone | Structure |
| Levonorgestrel | 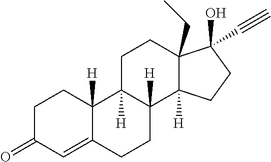 |
| Ethynyl Estradiol | 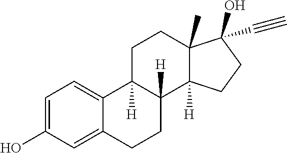 |
| Norethis- terone | 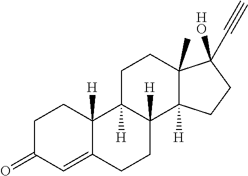 |
| Ethynodiol Diacetate | 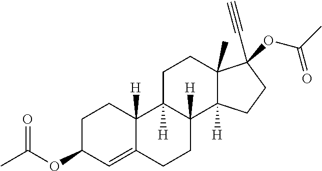 |
| Desogestrel | 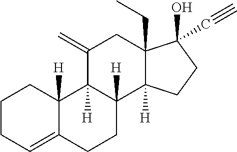 |
| Lynestrenol | 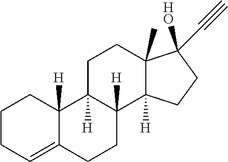 |
| Progesterone | 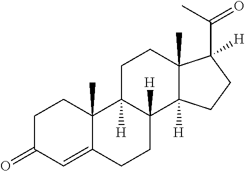 |

The present invention provides intravaginal drug delivery devices, such as intravaginal rings, comprising levonorgestrel, or other drugs having terminal alkene, alkyne or carbonyl functionalities, which exhibit increased recovery from platinum-catalyzed silicone polymers due to the optimization of drug particle size and cure conditions. Without intending to be bound by theory, it is believed that by decreasing the amount of drug solubilized in the silicone during the cure cycle, the amount of drug bound to the silicone polymer is reduced to an acceptable level for commercial drug products.

Specifically, the inventors of the instant invention have discovered that the amount of drug which is solubilized after mixing into the silicone system is determined by three factors: 1) total surface area of the drug exposed to the silicone polymer; 2) the temperature of the system, and 3) the time the drug has been in contact with the silicone. Thus, drug solubilization can be reduced by using highly crystalline drug material (so that more energy is needed to solubilize the drug), by using larger primary particle size, which reduces the surface area to increase solubilization time, and by reducing thermal exposure. By optimizing intravaginal ring formulations for drug particle size, cure temperature and cure time, the instant invention minimizes the amount of drug which binds to a silicone polymer during the platinum curing process, thereby resulting in higher recovery of the drug from platinum-catalyzed silicone intravaginal rings.

Various aspects of the invention are described in further detail in the following subsections:

### I. Definitions

As used herein, the term “silicone drug delivery device” refers to a device comprised of a silicone polymer which is designed to deliver a drug, or active pharmaceutical ingredient, to a subject to treat or prevent a disease or condition. Silicone drug delivery devices include, but are not limited to, intravaginal rings, diaphragms, cervical caps, and intrauterine devices (IUDs).

As used herein, the term “intrauterine device” or “IUD” refers to a device which is designed to be inserted into the uterus of a female human in order to provide controlled release of active pharmaceutical ingredients to the uterus over an extended period of time. IUDs often contain contraceptives for the purpose of providing long-acting reversible contraception to female humans. IUDs may also contain other active pharmaceutical ingredients for delivery to the vagina of a female human.

As used herein, the term “diaphragm” refers to a device which is designed to be inserted into the vagina of a female human in order to provide a seal against the walls of the vagina. Diaphragms may contain contraceptives for the purpose of providing reversible contraception to female humans. Diaphragms may also contain other active pharmaceutical ingredients for delivery to the vagina of a female human.

As used herein, the term “cervical cap” refers to a device which is designed to be inserted into the vagina of a female human to fit over the cervix. Cervical caps are often used as a contraceptive to block sperm from entering the uterus. Cervical caps may contain contraceptives for the purpose of providing reversible contraception to female humans. Cervical caps may also contain other active pharmaceutical ingredients for delivery to the vagina or cervix of a female human.

As used herein, the term “intravaginal ring” or “vaginal ring” refers to a toroid, or doughnut-shaped, polymeric drug delivery device which is designed to be inserted into the vagina of a female human in order to provide controlled release of drugs to the vagina over an extended period of time. Several single-indication intravaginal rings are currently commercially available, including Estring® and Femring®, for the treatment of symptoms of post-menopause, and NuvaRing®, a contraceptive vaginal ring. Other dual-indication intravaginal rings are currently being studied, including a dapivirine/levonorgestrel ring for the prevention of unwanted pregnancy and HIV transmission.

The intravaginal drug delivery devices of the instant invention provide controlled release of an active pharmaceutical ingredient (API) having a terminal alkene, alkyne or carbonyl group. In one embodiment, the API is a contraceptive. In one embodiment, the drug delivery device is an intravaginal ring, which provides controlled release of a contraceptive, such as levonorgestrel, alone or in combination with an antimicrobial compound or a second contraceptive. Intravaginal rings of the invention may have any shape and be of any dimensions compatible with intravaginal administration to a female human. Such a ring can be self-inserted into the vagina, where it is held in place due to its shape and inherent elasticity. Such a device provides high user adherence, ease of application and exhibits no leakage or messiness on insertion and subsequent placement within the vaginal space.

As used herein, the term “contraceptive” refers to an active agent that prevents conception or pregnancy. Contraceptives are well-known in the art and include, but are not limited to, 17a-ethinyl-levonorgestrel-17b-hydroxy-estra-4,9,11-trien-3-one, estradiol, etonogestrel, levonorgestrel (LNG), medroxyprogesterone acetate, nestorone, norethindrone, and progesterone. In one embodiment of the invention, the contraceptive is levonorgestrel (LNG), ethynyl estradiol, noresthisterone, ethynodiol diacetate, desogestrel, or lynestrenol. In one embodiment of the invention, the contraceptive is levonorgestrel. In one embodiment of the invention, the contraceptive is ethynyl estradiol. In one embodiment of the invention, the contraceptive is noresthisterone. In one embodiment of the invention, the contraceptive is ethynodiol diacetate. In one embodiment of the invention, the contraceptive is desogestrel. In one embodiment of the invention, the contraceptive is lynestrenol.

As used herein, the terms “compound having a terminal alkene, alkyne or carbonyl group”, “drug having a terminal alkene, alkyne or carbonyl group”, or “active pharmaceutical ingredient having a terminal alkene, alkyne or carbonyl group” refer to a compound having a terminal double bond, a terminal triple bond, or a terminal carbonyl group. Such compounds may include contraceptive agents and other active pharmaceutical ingredients. In one embodiment of the invention, the compound having a terminal alkene, alkyne or carbonyl group is a contraceptive agent. In another embodiment of the invention, the compound having a terminal alkene, alkyne or carbonyl group is an active pharmaceutical ingredient which is not a contraceptive. In one embodiment, the compound having a terminal alkene, alkyne or carbonyl group is an antiviral compound. In one embodiment, the compound having a terminal alkene, alkyne or carbonyl group is an antifungal compound. In one embodiment, the compound having a terminal alkene, alkyne or carbonyl group is an antimicrobial compound.

Contraceptive agents having a terminal alkene, alkyne or carbonyl group include, but are not limited to, levonorgestrel (LNG), ethynyl estradiol, noresthisterone, ethynodiol diacetate, desogestrel, or lynestrenol. In one embodiment of the invention, the contraceptive having a terminal alkene, alkyne or carbonyl group is levonorgestrel. In another embodiment of the invention, the contraceptive having a terminal alkene, alkyne or carbonyl group is ethynyl estradiol. In yet another embodiment of the invention, the contraceptive having a terminal alkene, alkyne or carbonyl group is noresthisterone. In a further embodiment of the invention, the contraceptive having a terminal alkene, alkyne or carbonyl group is ethynodiol diacetate. In one embodiment of the invention, the contraceptive having a terminal alkene, alkyne or carbonyl group is desogestrel. In one embodiment of the invention, the contraceptive having a terminal alkene, alkyne or carbonyl group is lynestrenol. In another embodiment, the contraceptive having a terminal alkene, alkyne or carbonyl group is progesterone.

As used herein, the term “levonorgestrel” or “LNG” refers to 13-ethyl-17-ethynyl-17-hydroxy-1,2,6,7,8,9,10,11,12,13,14,15,16,17-tetrad-ecahydrocyclopenta[a]phenanthren-3-one, a contraceptive compound. Levonorgestrel is a contraceptive agent that is useful in the prevention of pregnancy.

As used herein, the term “antimicrobial compound” or “antimicrobial agent” or “microbicide” (used interchangeably herein) refers to a compound or agent which is capable of inhibiting or destroying the growth of a microbial organism. In a preferred embodiment of the invention, the antimicrobial compound is dapivirine. In another preferred embodiment, the antimicrobial compound is a non-nucleoside reverse transcriptase inhibitor (“NNRTI”). In another embodiment, the NNRTI is a substituted di-amino pyrimidine derivative. In another embodiment, the antimicrobial compound is a viral entry inhibitor. In one embodiment of the invention, the antimicrobial compound is maraviroc. In one embodiment of the invention, the antimicrobial is DS003. In another embodiment of the invention, the antimicrobial compound is darunavir, GSK 1265744 or BMS-663068.

The term “antimicrobial compound” is intended to embrace antibacterial agents, antifungal agents, antiprotozoal agents, antiviral agents and mixtures thereof. The antiviral agents darunivir, atazanavir, ritonavir, emtricitabine, zidovudine, maraviroc, lopinavir, lamivudine, and fosamprenavir all have a terminal carbonyl bond. In one embodiment, the antiviral agent is Darunivir. In one embodiment, the antiviral agent is atazanavir. In one embodiment, the antiviral agent is ritonavir. In one embodiment, the antiviral agent is emtricitabine. In one embodiment, the antiviral agent is zidovudine. In one embodiment, the antiviral agent is lopinavir. In one embodiment, the antiviral agent is lamivudine. In one embodiment, the antiviral agent is fosamprenavir. In one embodiment, the antifungal agent is ketoconazole.

As used herein, the term “dapivirine” refers to (4-[[4-[(2,4,6-trimethylphenyl)amino]-2-pyrimidinyl]amino]benzonitrile), a non-nucleoside reverse transcriptase inhibitor. Dapivirine is useful in the prevention and/or treatment of retroviral infection, such as HIV-1 infection. Dapivirine is a crystalline compound that is white to slightly beige in color, has a melting point of about 220° C. and is virtually insoluble in water. More specifically, the solubility of dapivirine is less than 0.001 mg/mLf of water (i.e., less than 1 μg/ml of water). The intravaginal rings of the instant invention may use micronized dapivirine. A composite result (four samples taken of micronized material) showed that 88.15% of the material had a particle size of less than 5 microns (μM).

As used herein, the term “matrix ring” or “matrix-type ring” refers to an intravaginal ring in which the active agent or agents are homogenously distributed throughout the ring. Matrix rings are typically manufactured by injection molding or extrusion of a compound-containing active mix, leading to the uniform distribution of the active compound throughout the ring. The matrix-type rings of the instant invention may comprise a contraceptive agent, alone or in combination with another contraceptive agent and/or an antimicrobial agent, dispersed in silicone elastomer with normal propylorthosilicate (NPOS) crosslinker. This active mix is subsequently cured using a catalyst, such as platinum (with curing achieved by an addition reaction). Matrix-type intravaginal rings permit single intravaginal dosing of active agent(s), with an initially high “loading” and a subsequent, lower “maintenance” release profile.

As used herein, the term “platinum-catalyzed” refers to an intravaginal ring whose cross-linking reaction has been catalyzed using an organo-platinum compound. In one embodiment, the intravaginal ring comprises a silicone elastomer. In yet another embodiment, the intravaginal ring comprises a silicone elastomer and a silicone dispersant. The intravaginal ring may comprise other pharmaceutically compatible agents. Such agents include pharmacologically active agents, as well as, pharmacologically inactive agents known in the art as pharmaceutical excipients.

As used herein, the term “reservoir ring” refers to an intravaginal ring comprising a reservoir (a full or partial-length core), which is completely surrounded by a sheath. The release of drug substances from such rings is dependent upon permeation (i.e., molecular dissolution and subsequent diffusion) of the core-loaded drug substance through the outer sheath. Additionally, the drug substance may be loaded into the core, the sheath, or both. Release rates can be modified by changing the nature or thickness of the rate-controlling sheath. Reservoir rings were developed to provide controlled (that is, constant daily) release rates.

In one embodiment, the compound having a terminal alkene, alkyne or carbonyl group is present in the core of a reservoir ring, with a blank sheath. In one embodiment, the core of a reservoir ring can comprise the compound having a terminal alkene, alkyne or carbonyl group and at least one additional compound having a terminal alkene, alkyne or carbonyl group, with a blank sheath. In another embodiment, the core of a reservoir ring can comprise the compound having a terminal alkene, alkyne or carbonyl group and an additional antimicrobial agent, with a blank sheath.

In another embodiment, a compound having a terminal alkene, alkyne or carbonyl group is present in a half-core (or partial core) of a reservoir ring, with a blank sheath. In one embodiment, the compound having a terminal alkene, alkyne or carbonyl group can be present in a half-core of a reservoir ring, and an antimicrobial compound can be present in the other half-core of the reservoir ring, with a blank sheath. In another embodiment, the compound having a terminal alkene, alkyne or carbonyl group can be present in a half-core of a reservoir ring, and a different compound having a terminal alkene, alkyne or carbonyl group can be present in the other half-core of the reservoir ring, with a blank sheath.

In yet another embodiment, a compound having a terminal alkene, alkyne or carbonyl group is present in the core of a reservoir ring, and an antimicrobial agent is present in the sheath. In another embodiment, an antimicrobial agent is present in the core of a reservoir ring, and the compound having a terminal alkene, alkyne or carbonyl group is present in the sheath. In yet another embodiment, a compound having a terminal alkene, alkyne or carbonyl group is present in the core of a reservoir ring, and a different compound having a terminal alkene, alkyne or carbonyl group is present in the sheath.

As used herein, the term “micronize” refers to a process of reducing the average diameter of a solid material's particles. As used herein, the term “micronized” refers to a drug or compound which has undergone a process of reducing the average diameter of the solid drug or compound's particles. Typical micronization techniques are well-known in the art and utilize friction to reduce particle size and include milling, bashing, grinding, and fluidizing. Other micronization methods are well known in the art, and include RESS (Rapid Expansion of Supercritical Solutions), SAS (Supercritical Anti-Solvent) and PGSS (Particles from Gas Saturated Solutions). Typically, a compound having a d90 (diameter at which 90% of the sample's measured particles are smaller sized particles) of less than 25 is considered “micronized.”

As used herein, the term “non-micronized” refers to particles which have not undergone a process of reducing the average diameter of the particles.

As used herein, the term “bound” refers to an attraction between two or more atoms that allows the formation of a chemical substance that contains the two or more atoms. A compound, e.g., a contraceptive, having a terminal alkene, alkyne or carbonyl group can be “bound” to a silicone hydride group of a silicone polymer via a covalent bond. In one embodiment of the invention, a compound of the invention may be covalently bound to a silicone hydride group of a silicone polymer via a terminal double (alkene) bond (C═C). In one embodiment of the invention, a compound of the invention may be covalently bound to a silicone hydride group of a silicone polymer via a terminal tripe (alkyne) bond (C≡C). In one embodiment of the invention, a compound of the invention may be covalently bound to a silicone hydride group of a silicone polymer via a terminal carbonyl group (C═O). In one embodiment, the terminal carbonyl is a ketone. In another embodiment, the terminal carbonyl is an α,β-unsaturated ketone.

As used herein, the term “recoverable” or “recovery” refers to the ability of a compound having a terminal alkene, alkyne or carbonyl group to be released from a platinum-catalyzed silicone intravaginal drug delivery device, e.g., intravaginal ring, of the invention. Compounds having a terminal alkene, alkyne or carbonyl group that are recoverable from the intravaginal drug delivery devices of the invention are not covalently bound to the silicone polymer of the device. Rather, compounds, e.g., contraceptives, that are recoverable from the intravaginal drug delivery devices of the invention are incorporated into the device and able to be released from the intravaginal drug delivery device. Recovery of compounds, e.g., contraceptives, from intravaginal drug delivery devices of the invention is typically defined by percentages. For example, at least 75% of the contraceptive is recoverable from the intravaginal drug delivery device, e.g., intravaginal ring.

It is important to note that the “release rate” of a compound having a terminal alkene, alkyne or carbonyl group from the intravaginal drug delivery device is completely distinct, and separate from, the percentage of compound that is “recoverable” from the device, e.g., intravaginal ring. For example, a compound having a terminal alkene, alkyne or carbonyl group may be released at a fast rate from an intravaginal ring, but yet have a low percent recovery. Alternatively, a compound having a terminal alkene, alkyne or carbonyl group may be released at a slow rate from an intravaginal ring, but yet have a high percent recovery. In another alternative, a compound having a terminal alkene, alkyne or carbonyl group may be released at a slow rate and have a low percent recovery, or may be released at a fast rate and have a high percent recovery.

As used herein, the term “release” or “release rate” refers to the amount or concentration of compound having a terminal alkene, alkyne or carbonyl group, e.g., contraceptive, which leaves the platinum-catalyzed silicone intravaginal drug delivery device, e.g., intravaginal ring, in any defined time period. “Sustained release” or “sustained release rate” refers to release sufficient to provide active pharmaceutical ingredient properties, e.g., contraceptive properties, over a specific time period. Release rates of the compounds, e.g., contraceptives, are defined in more detail in the subsections, below. For example, in one embodiment of the invention, the intravaginal rings are designed to provide sustained release of the compound, e.g., contraceptive. In one embodiment of the invention, the contraceptive having a terminal alkene, alkyne or carbonyl group, such as levonorgestrel, is released at a range of 800-1000 μg within the first 24 hours, and then >70 μg each day for up to 89 days after the initial 24 hour period of release.

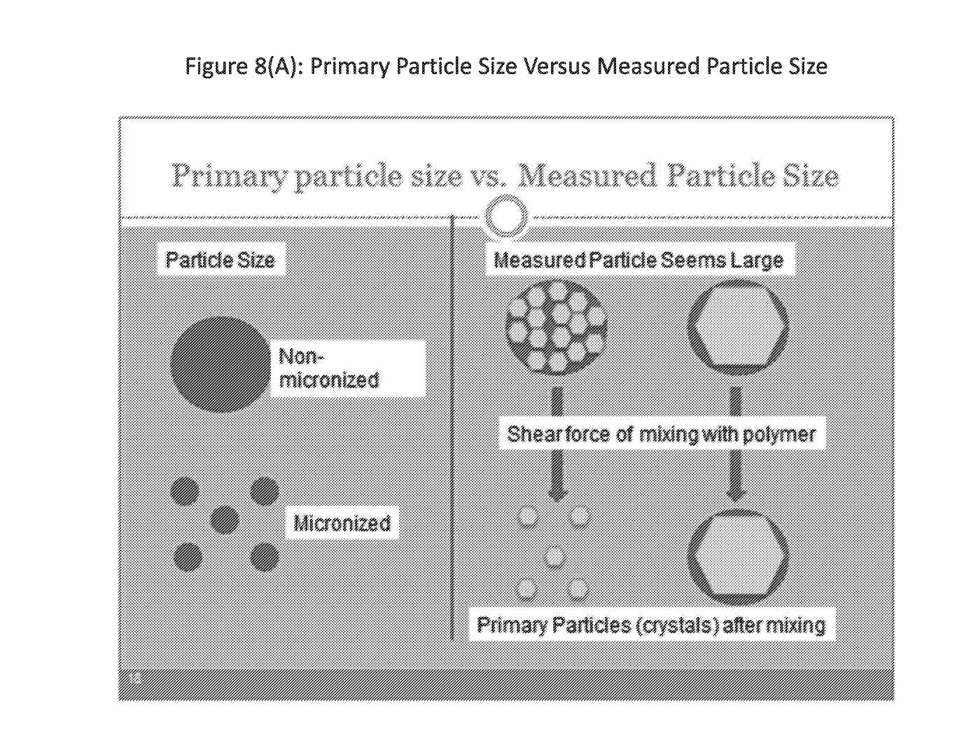

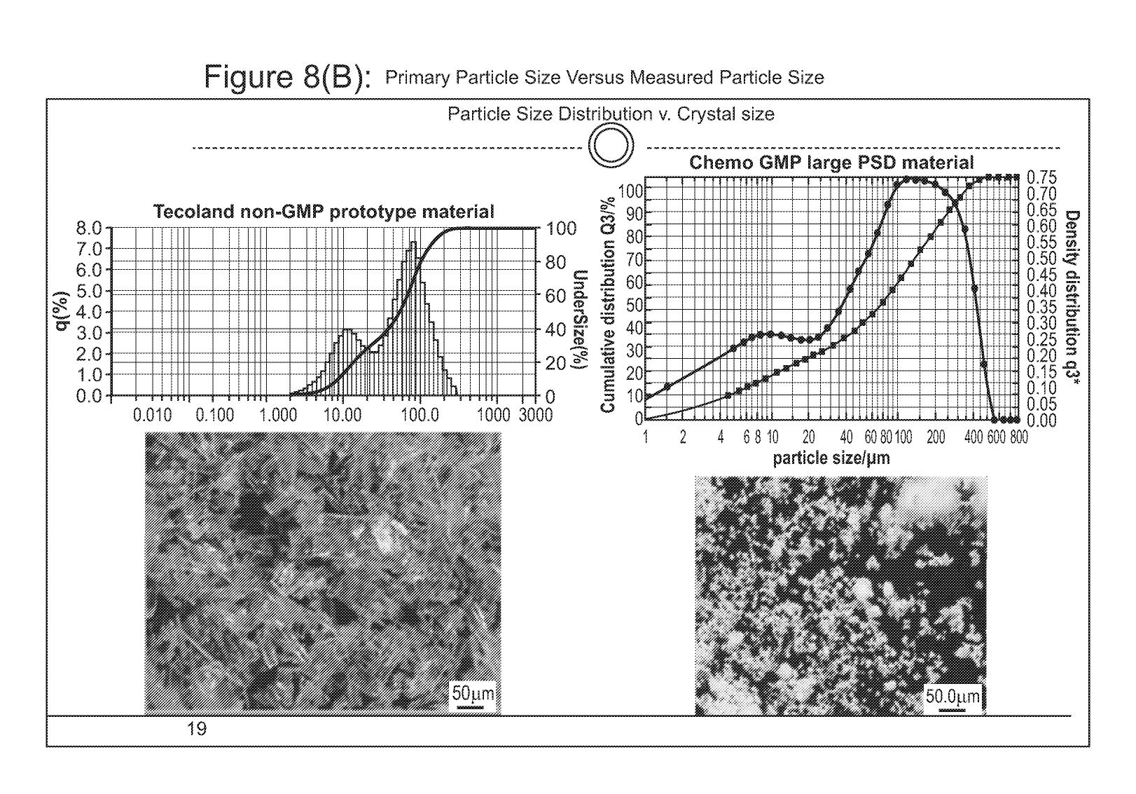

As used herein, the term “primary particle size” refers to the average size of the smallest particle of a compound having a terminal alkene, alkyne or carbonyl group of the invention. Primary particle size is often defined by the d50, diameter at which 50% of the sample's measured particles are smaller sized particles, or by the d90, diameter at which 90% of the sample's measured particles are smaller sized particles. d50 and d90 values are commonly determined by one of ordinary skill in the art using various techniques, such as laser diffraction, dynamic light scattering, electrophoretic light scattering, automated imaging, sedimentation, electrozone sensing, light obscuration, image analysis, and sieving. Primary particles often agglomerate, aggregate, or cluster into larger structures comprising multiple primary particles (see, for example FIG. 8(A)).

### II. Intravaginal Rings

The present invention provides platinum-catalyzed silicone intravaginal drug delivery devices, e.g., intravaginal rings, useful for the administration of therapeutic and/or prophylactic agents to a human. The intravaginal rings of the invention may provide long-term controlled release of a compound having a terminal alkene, alkyne or carbonyl group, e.g., a contraceptive, such as levonorgestrel, ethynyl estradiol, noresthisterone, ethynodiol diacetate, desogestrel or lynestrenol. In some embodiments, the intravaginal rings of the invention may provide long-term controlled release of a compound having a terminal alkene, alkyne or carbonyl group, e.g., a contraceptive, and an antimicrobial agent, such as dapivirine, maraviroc, DS003, darunavir, GSK1265744 or BMS-663068. Surprisingly, the inventors of the instant application have discovered a method for optimizing curing conditions of platinum-catalyzed silicone drug delivery devices, e.g., intravaginal rings, that result in decreased binding of compounds having terminal alkene, alkyne or carbonyl groups to the silicone and increased recovery of the compound from the intravaginal ring to levels not previously obtainable before the instant invention.

As used herein, the term “intravaginal ring” or “vaginal ring” refers to a doughnut-shaped polymeric drug delivery device which is designed to be inserted into the vagina of a female human in order to provide controlled release of drugs to the vagina over an extended period of time. Several single-indication intravaginal rings are currently available, including Estring® and Femring®, for the treatment of urogenital symptoms of post-menopause, and NuvaRing®, a contraceptive vaginal ring. Intravaginal rings are described in U.S. Pat. No. 6,951,654, U.S. Patent Application Publication Nos. US2007/0043332 and US2009/0004246, PCT Publication Nos. WO99/50250, WO02/076426 and WO03/094920, the entire contents of each of which are expressly incorporated herein by reference.

The intravaginal rings of the instant invention may provide controlled release of a contraceptive, alone or in combination with an antimicrobial compound, and may have any shape and be of any dimensions compatible with intravaginal administration to a female human. Such a ring can be self-inserted into the vagina, where it is held in place due to its shape and inherent elasticity. In one embodiment, the intravaginal ring has an outer diameter of 56 mm. In another embodiment, the intravaginal ring has an outer diameter of about 50 mm, about 51 mm, about 52 mm, about 53 mm, about 54 mm, about 55 mm, about 56 mm, about 57 mm, about 58 mm, about 59 mm or about 60 mm. In another embodiment, the intravaginal ring has a cross-sectional diameter of 7.7 mm. In yet another embodiment, the intravaginal ring has a cross-sectional diameter of about 7.0 mm, about 7.1 mm, about 7.2 mm, about 7.3 mm, about 7.4 mm, about 7.5 mm, about 7.6 mm, about 7.7 mm, about 7.8 mm, about 7.9 mm, about 8.0 mm, about 8.1 mm, about 8.2 mm, about 8.3 mm, about 8.4 mm, or about 8.5 mm.

Such an intravaginal ring permits single intravaginal dosing of a contraceptive, or a contraceptive in combination with an antimicrobial agent, with a stable release profile. In addition, a device that can be applied less frequently is likely be more acceptable and to achieve better adherence relative to gels that need to be used more frequently

The intravaginal rings of the invention comprise a silicone elastomer. In one embodiment, the intravaginal ring comprises a silicone elastomer and a silicone dispersant.

The intravaginal ring may comprise other pharmaceutically compatible agents. Such agents include pharmacologically active agents, as well as, pharmacologically inactive agents known in the art as pharmaceutical excipients. Examples of pharmacologically active agents that may be advantageous include, but are not limited to, a local anesthetic such as lidocaine or a local analgesic or a mixture thereof. Examples of pharmacologically inactive agents that may be advantageous include, but are not limited to, a buffer (or buffers), or hydrophilic compounds that enhance the rate of release of the agent from the device, such as for example, polyvinylpyrrolidone (PVP or povidone), modified cellulose ethers (e.g., hydroxyethylcellulose, hydroxypropylcellulose and hydroxypropylmethylcellulose) microcrystalline cellulose, polyacrylic acid, carbomer, alginic acid, carrageenan, cyclodextrins, dextrin, guar gum, gelatin, xanthan gum and sugars (e.g., monosaccharides such as glucose, fructose and galactose, and dissaccharides such as lactose, maltose and fructose). When employed, the release rate enhancing excipient is generally present in an amount of about 0.5 to about 40 w/w % and preferably about 2.5 to about 15 w/w % of the device.

As used herein, the term “matrix ring” or “matrix-type ring” refers to an intravaginal ring in which a contraceptive, or a contraceptive and an antimicrobial agent, are homogenously distributed throughout the ring. Matrix rings are typically manufactured by injection molding or extrusion of the active compound-containing active mix, leading to the uniform distribution of the active compounds throughout the ring. The matrix-type rings of the instant invention may comprise a contraceptive dispersed in silicone elastomer with normal propylorthosilicate (NPOS) crosslinker. This active mix is subsequently cured using a catalyst, such as platinum (with curing achieved by an addition reaction). The matrix-type rings of the invention do not comprise a polyurethane or EVA polymer. The matrix-type rings of the invention are also not cured with tin catalysts.

As used herein, the term “reservoir ring” refers to an intravaginal ring comprising a reservoir (a full or partial-length core), which is completely surrounded by a sheath. The release of drug substances from such rings is dependent upon permeation (i.e., molecular dissolution and subsequent diffusion) of the core-loaded drug substance through the outer sheath. Release rates can be modified by changing the nature or thickness of the rate-controlling sheath. Reservoir rings were developed to provide controlled (that is, constant daily) release rates.

As used herein, the term “elastomer” refers to an amorphous, or predominantly amorphous, polymer network formed when a polymer or a mixture of polymers undergo cross-linking. Each polymer is comprised of monomeric units, which are linked together to form the network. The monomeric units can comprise carbon, hydrogen, oxygen, silicon, halogen, or a combination thereof.

The intravaginal rings of the invention comprise a polysiloxane. As used herein, a “polysiloxane” refers to any of various compounds containing alternate silicon and oxygen atoms in either a linear or cyclic arrangement usually with one or two organic groups attached to each silicon atom. For example, polysiloxanes include substituted polysiloxanes, and diorganopolysiloxanes such as diarylpolysiloxanes and dialkylpolysiloxanes; an example of the latter is dimethylpolysiloxane. Such dimethylpolysiloxane polymers can be thermoset to the corresponding elastomer by vulcanization with peroxide curing catalysts, e.g., benzoyl peroxide or di-p-chlorobenzoyl peroxide at temperatures of about 200° C. and requiring additional heat after treatment as described in U.S. Pat. Nos. 2,541,137; 2,723,966; 2,863,846; 2,890,188; and 3,022,951, the entire contents of each of which are expressly incorporated herein by reference.

An example of a two-component silicone elastomer, which is platinum-catalyzed at room temperature or under slightly elevated temperature and capable of cross-linking, is NuSil MED-4870 (NuSil Technology LLC, Carpinteria, Calif.). In some embodiments of the present invention, an intravaginal ring can comprise silicone liquid (NuSil MED360) as a dispersing agent, and NuSil MED-4870 elastomer. The NuSil MED-4870 elastomer is composed of two parts, part A and part B. The chemical composition of NuSil MED-4870 part A comprises vinyl terminated polydimethylsiloxane (linear) polymers as a polymer, platinum-siloxane complex as the catalyst for the cross-linking reaction, and ˜30% amorphous (non crystalline) reinforcing silica as a filler. The chemical composition of NuSil MED-4870 part B comprises vinyl-terminated polydimethylsiloxane (linear) polymers, hydride functional polydimethysiloxane polymer as a cross-linker, and ˜30% amorphous (non-crystalline) reinforcing silica as a filler. Form A and form B undergo cross-linking to form a silicone elastomer.

In some embodiments of the present invention, the polysiloxane elastomer is a diorganopolysiloxane elastomer. In some embodiments, the diorganopolysiloxane elastomer is dimethylpolysiloxane elastomer. In some embodiments, the dimethylpolysiloxane elastomer further comprises a dimethylmethylhydrogen polysiloxane cross-link. In some embodiments of the present invention, the polysiloxane elastomer is NuSil MED-4870. In another embodiment, the silicone elastomer is NuSil DDU-4320. In another embodiment, the silicone elastomer is MED-4320. In one embodiment, the polysiloxane elastomer can be purchased from a manufacturer who provides medical grade implantable liquid silicone rubbers (LSRs), such as Applied Silicones, Bluestar Technologies, Dow Corning, Wacker, Momentive, or other suppliers.

In some embodiments, the polysiloxane elastomer is present in a concentration of about 90% to about 99% by total weight of the ring. In some embodiments, the polysiloxane elastomer is present in a concentration of about 95% by total weight of the ring, or about 97% by total weight of the ring.

Suitable cross-linking agents and curing catalysts are well known in the art. Curing temperatures and times will vary, depending on the particular elastomer(s) used. For example, the curing temperature may vary between room temperature (15-25° C.) and 160° C. but is preferably within the range 60-200° C. The curing time may vary between a few seconds and several hours, depending on the elastomer(s) used. Preferred and suitable elastomers include two-component dimethylpolysiloxane compositions using platinum as the curing catalyst and at a curing temperature of from room temperature to an elevated temperature.

As used herein, the term “platinum-catalyzed” refers to an intravaginal ring whose cross-linking reaction has been catalyzed using an organo-platinum compound.

As used herein, the term “recoverable” or “recovery” refers to the ability of a compound having a terminal alkene, alkyne or carbonyl group to be released from a platinum-catalyzed silicone intravaginal drug delivery device, e.g., intravaginal ring, of the invention. Compounds having a terminal alkene, alkyne or carbonyl group that are recoverable from the intravaginal drug delivery device of the invention are not covalently bound to the silicone polymer of the intravaginal drug delivery device. Rather, compounds, e.g., contraceptives, which are recoverable from the intravaginal drug delivery devices of the invention are incorporated into the intravaginal drug delivery device and able to be released from the intravaginal drug delivery device. Recovery of compounds, e.g., contraceptives, from intravaginal drug delivery devices, e.g., intravaginal rings, of the invention is typically defined by percentages. For example, at least 75% of the compound, e.g., contraceptive, is recoverable from the intravaginal drug delivery device.

More specifically, and without wishing to be bound by mechanism, about 99% of a compound of the invention may be recoverable from an intravaginal ring of the invention, for example, if about 1% of the compound is covalently bound to the silicone polymer of the intravaginal ring. In another embodiment, about 95% of a compound of the invention may be recoverable from an intravaginal ring of the invention, for example, if about 5% of the compound is covalently bound to the silicone polymer of the intravaginal ring. In another embodiment, about 90% of a compound of the invention may be recoverable from an intravaginal ring of the invention, for example, if about 10% of the compound is covalently bound to the silicone polymer of the intravaginal ring. In another embodiment, about 80% of a compound of the invention may be recoverable from an intravaginal ring of the invention, for example, if about 20% of the compound is covalently bound to the silicone polymer of the intravaginal ring. In another embodiment, about 75% of a compound of the invention may be recoverable from an intravaginal ring of the invention, for example, if about 25% of the compound is covalently bound to the silicone polymer of the intravaginal ring.

It is important to note that the “release rate” of a compound having a terminal alkene, alkyne or carbonyl group from the intravaginal rings is completely distinct, and separate from, the percentage of compound that is “recoverable” from the intravaginal ring. For example, a compound having a terminal alkene, alkyne or carbonyl group may be released at a fast rate from an intravaginal ring, but yet have a low percent recovery. Alternatively, a compound having a terminal alkene, alkyne or carbonyl group may be released at a slow rate from an intravaginal ring, but yet have a high percent recovery. In another alternative, a compound having a terminal alkene, alkyne or carbonyl group may be released at a slow rate and have a low percent recovery, or may be released at a fast rate and have a high percent recovery.

As an example, 32 mg of a compound having a terminal alkene, alkyne or carbonyl group loaded into a platinum-catalyzed silicone intravaginal ring may be released at a rate of about 1 mg during the first 24 hours, then at a rate of about 70 μg per day for two weeks afterwards, and then the release rate falls to about 0 μg per day. However, only a total of about 1.98 mg of the 32 total mg of the compound was recoverable from the intravaginal ring because the release rate of the contraceptive from the ring after week two fell to 0 μg per day. Thus, the release rates differ from, and are independent of, the percentage of compound having a terminal alkene, alkyne or carbonyl group that is recoverable from the ring.

As used herein, the term “release” or “release rate” refers to the amount or concentration of active pharmaceutical ingredient (e.g., compound having a terminal alkene, alkyne or carbonyl group) which leaves the intravaginal ring in any defined time period. “Sustained release” or “sustained release rate” refers to release sufficient to provide antimicrobial properties or contraceptive properties over a specific time period. Release rates of the contraceptive and the antimicrobial agent are defined in more detail in the subsections, below. For example, in one embodiment of the invention, the intravaginal rings are designed to provide sustained release of the contraceptive. In a preferred embodiment, the contraceptive, such as levonorgestrel, is released at a range of 800-1000 μg within the first 24 hours, and then >70 μg each day for up to 89 days after the initial 24 hour period of release. In one embodiment, the intravaginal ring is designed to provide sustained release of a contraceptive and an antimicrobial agent. In a preferred embodiment, the antimicrobial, such as dapivirine, is released at a rate of about 200 μg per day.

As used herein, the term “controlled release rate” refers to a constant release rate that can be determined by the design and drug loading of the vaginal ring.

As used herein, the term “constant release rate” refers to a release rate which does not readily change with device storage over time. Preferably, the release rate of the contraceptive and/or the antimicrobial agent from the intravaginal ring is constant, or stable and does not readily change over time at room temperature (about 30° C.) or at 40° C. for at least 1 month, at about 2-8° C. for at least 1 year, or for at least 2 years. For example, the release rate of the contraceptive or the antimicrobial agent from the intravaginal rings of the instant invention can be stable for 1, 2, 3, 4, 5, 6, 7, 8, 9, 10, 11, 12, 13, 14, 15, 16, 17, 18, 19, 20, 21, 22, 23, 24, 30, 36, 42 or 48 months.

As used herein, the term “steady release rate” means a release rate that shows relatively little change over time.

As used herein, the term “initial 24 hour period of use” refers to the first day, or twenty-four hours, of time after the initial use of the intravaginal ring. The initial 24 hour period of use begins when the intravaginal ring is inserted into the vagina of the female human.

As used herein, the term “each day” refers to an individual 24 hour period.

As used herein, the term “homogenously dispersed throughout” refers to a contraceptive compound or an antimicrobial compound which is uniformly distributed throughout the intravaginal ring.

As used herein, the term “prophylactically effective amount” refers to the amount of compound effective to prevent development of a condition or a disease in the subject. In one embodiment, the condition is conception or pregnancy. In another embodiment of the invention, the disease is HIV.

As used herein, the term “therapeutically effective amount” refers to the amount of compound effective to treat a disease or condition in a subject. In one embodiment of the invention, the disease is HIV.

A “stable” compound is one which essentially retains its physical stability and/or chemical stability and/or biological activity during the manufacturing process and/or upon storage. Various analytical techniques for measuring stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993).

As used herein, the term “storage” refers to the period of time after which the intravaginal rings are made, but before which the intravaginal rings are used. For example, the intravaginal rings of the instant invention can be stored for 1, 2, 3, 4, 5, 6, 7, 8, 9, 10, 11, 12, 13, 14, 15, 16, 17, 18, 19, 20, 21, 22, 23, 24, 30, 36, 42 or 48 months.

### III. Contraceptives Having a Terminal Alkene, Alkyne or Carbonyl Group

The present invention provides intravaginal drug delivery devices comprising a compound having a terminal alkene, alkyne or carbonyl group. In one embodiment, the compound is a contraceptive, such as levonorgestrel, which exhibits increased recovery from platinum-catalyzed silicone polymers due to the optimization of drug particle size and cure conditions.

As used herein, the term “contraceptive” refers to an active pharmaceutical ingredient that prevents conception or pregnancy. Contraceptives are well-known in the art and include, but are not limited to, steroid hormones and include, for example, an estrogen, a progestin, a progesterone, a testosterone, derivatives thereof, or combinations thereof. Examples of contraceptives include 17a-ethinyl-levonorgestrel-17b-hydroxy-estra-4,9,11-trien-3-one, estradiol, etonogestrel, levonorgestrel, ethynyl estradiol, noresthisterone, ethynodiol diacetate, desogestrel, lynestrenol, medroxyprogesterone acetate, nestorone, norethindrone, and progesterone. In one embodiment of the invention, the contraceptive is levonorgestrel.

As used herein, the term “contraceptive comprising a terminal alkene or a terminal alkyne” refers to a contraceptive having a terminal double bond, triple bond and/or carbonyl group. Such contraceptive agents include, but are not limited to, levonorgestrel (LNG), ethynyl estradiol, noresthisterone, ethynodiol diacetate, desogestrel, or lynestrenol. In one embodiment of the invention, the contraceptive is levonorgestrel. In one embodiment of the invention, the contraceptive is ethynyl estradiol. In one embodiment of the invention, the contraceptive is noresthisterone. In one embodiment of the invention, the contraceptive is ethynodiol diacetate. In one embodiment of the invention, the contraceptive is desogestrel. In one embodiment of the invention, the contraceptive is lynestrenol. In another embodiment, the contraceptive is progesterone.

As used herein, the term “levonorgestrel” or “LNG” refers to 13-ethyl-17-ethynyl-17-hydroxy-1,2,6,7,8,9,10,11,12,13,14,15,16,17-tetrad-ecahydrocyclopenta[a]phenanthren-3-one, a contraceptive compound. Levonorgestrel is useful in the prevention of pregnancy.

As used herein, a “progestin” refers to a progestogen, a progestational substance, or any pharmaceutically acceptable substance in the steroid art that generally possesses progestational activity including synthetic steroids that have progestational activity. Progestins suitable for use with the present invention can be of natural or synthetic origin. Progestins generally possess a cyclo-pentanophertanthrene nucleus.

Progestins suitable for use in the present invention include, but are not limited to, natural and synthetic compounds having progestational activity, such as, for example, progesterone, medroxyprogesterone, medroxyprogesterone acetate, chlormadinone acetate, norethindrone, cyproterone acetate, norethindrone acetate, desogestrel, levonorgestrel, drospirenone, trimegestone, norgestrel, norgestimate, norelgestromin, etonogestrel, dienogest, gestodene, megestrol, and other natural and/or synthetic gestagens. In some embodiments, the progestin is progesterone, etonogestrel, levonorgestrel, gestodene, norethisterone, drospirenone, or combinations thereof. In one embodiment, the progestin is levonorgestrel. In another embodiment, the progestin is nesterone.

Prodrugs of suitable progestins can also be used in the intravaginal device of the present invention. Ethynodiol diacetate, which is converted in vivo to norethindrone, is an example of a progestin prodrug that can be used in the present invention. Additional examples of progestin prodrugs include, but are not limited to, norgestimate (which is converted in vivo to 17-deacetyl norgestimate, also known as norelgestromin), desogestrel (which is converted in vivo to 3-keto desogestrel, also known as etonogestrel), and norethindrone acetate (which is converted in vivo to norethindrone).

In some embodiments, the intravaginal ring of the present invention may comprise one or more contraceptive agents. In some embodiments, the intravaginal ring of the invention comprises at least two, three, four, five or six contraceptive agents. In some embodiments, the intravaginal ring comprises a contraceptive and an antimicrobial compound, such as dapivirine.

In one embodiment, about 10 to about 30 mg of contraceptive is present in the ring. In another embodiment, about 20 mg to about 30 mg of contraceptive is present in the ring. In yet another embodiment, about 10 to about 800 mg, about 50 mg to about 750 mg, about 100 mg to about 700 mg, or about 200 mg to about 600 mg, about 300 mg to about 400 mg of contraceptive is present in the ring.

In another embodiment, about 15 mg of contraceptive is present in the ring. In another embodiment, about 16 mg of contraceptive is present in the ring. In another embodiment, about 25 mg of contraceptive is present in the ring. In another embodiment, about 32 mg of contraceptive is present in the ring. In another embodiment, about 64 mg of the contraceptive is present in the ring. In another embodiment, about 100 mg of contraceptive is present in the ring. In another embodiment, about 150 mg of contraceptive is present in the ring. In another embodiment, about 250 mg of contraceptive is present in the ring. In another embodiment, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 55 mg, about 60 mg, about 65 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 350 mg, about 400 mg, about 450 mg, about 500 mg, about 550 mg, about 600 mg, about 650 mg, about 700 mg, about 750 mg, or about 800 mg of contraceptive is present in the ring.

In one embodiment, at least about 75% of the contraceptive is recoverable from the ring. In another embodiment, at least about 80% of the contraceptive is recoverable from the ring. In another embodiment, at least about 85% of the contraceptive is recoverable from the ring. In another embodiment, at least about 90% of the contraceptive is recoverable from the ring. In another embodiment, at least about 95% of the contraceptive is recoverable from the ring. In another embodiment, about 99% of the contraceptive is recoverable from the ring. In another embodiment, about 100% of the contraceptive is recoverable from the ring. In another embodiment, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the contraceptive is recoverable from the ring. In a further embodiment, at least about 97.5%, 97.6%, 97.7%, 97.8%, 97.9%, 98.05, 98.1%, 98.2%, 98.3%, 98.4%, 98.5%, 98.6%, 98.7%, 98.8%, 98.9%, 99.05, 99.1%, 99.2%, 99.3%, 99.4%, 99.5%, 99.6%, 99.7%, 99.8%, or 99.9% of the contraceptive is recoverable from the ring.

In one embodiment, less than about 25% of the contraceptive is covalently bound to the silicone. In one embodiment, less than about 20% of the contraceptive is covalently bound to the silicone. In one embodiment, less than about 15% of the contraceptive is covalently bound to the silicone. In one embodiment, less than about 10% of the contraceptive is covalently bound to the silicone. In one embodiment, less than about 5% of the contraceptive is covalently bound to the silicone. In one embodiment, less than about 1% of the contraceptive is covalently bound to the silicone. In one embodiment, about 0% of the contraceptive is covalently bound to the silicone. In another embodiment, less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% of the contraceptive is covalently bound to the silicone. In a further embodiment, less than about 2.5%, 2.4%, 2.3%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the contraceptive is covalently bound to the silicone. In yet another embodiment, the amount of contraceptive covalently bound to the silicone is below the limit of detection.

In one embodiment, less than about 2 mg of the contraceptive is released from the ring in vitro during an initial 24 hour period of release. In another embodiment, less than about 1 mg of the contraceptive is released from the ring in vitro during an initial 24 hour period of release. In one embodiment, the contraceptive is released in vitro at a rate of about 20 μg per day to about 290 μg per day for about 23 days after an initial 7 day period of release. In another embodiment, the contraceptive is released in vitro at a rate of about 20 μg per day to about 290 μg per day for about 53 days after the initial 7 day period of release. In another embodiment, the contraceptive is released in vitro at a rate of about 35 μg per day to about 70 μg per day for about 23 days after the initial 7 day period of release. In another embodiment, the contraceptive is released in vitro at a rate of about 35 μg per day to about 70 μg per day for about 53 days after the initial 7 day period of release. In another embodiment, the contraceptive is released in vitro at a rate of about 35 μg per day to about 70 μg per day for about 83 days after the initial 7 day period of release.

In one embodiment, less than about 100 μg per day of the contraceptive is released in vitro after the initial 7 day period of release for about 23 days. In another embodiment, less than about 100 μg per day of the contraceptive is released in vitro after the initial 7 day period of release for about 53 days. In another embodiment, less than about 100 μg per day of the contraceptive is released in vitro after the initial 7 day period of release for about 83 days. In one embodiment, less than about 70 μg per day of the contraceptive is released in vitro after the initial 7 day period of release for about 23 days. In one embodiment, less than about 70 μg per day of the contraceptive is released in vitro after the initial 7 day period of release for about 53 days. In one embodiment, less than about 70 μg per day of the contraceptive is released in vitro after the initial 7 day period of release for about 83 days. In one embodiment, less than about 15 μg per day of the contraceptive is released in vitro after the initial 7 day period of release for about 23 days. In one embodiment, less than about 15 μg per day of the contraceptive is released in vitro after the initial 7 day period of release for about 53 days. In one embodiment, less than about 15 μg per day of the contraceptive is released in vitro after the initial 7 day period of release for about 83 days. In one embodiment, about 35 μg per day of the contraceptive is released in vitro after the initial 7 day period of release for about 23 days or for about 53 days. In one embodiment, about 35 μg per day of the contraceptive is released in vitro after the initial 7 day period of release for about 23 days or for about 83 days.

In one embodiment, the contraceptive is released in vitro at a rate of about 20 μg per day to about 290 μg per day for about 30 days. In another embodiment, the contraceptive is released in vitro at a rate of about 20 μg per day to about 290 μg per day for about 60 days. In another embodiment, the contraceptive is released in vitro at a rate of about 20 μg per day to about 290 μg per day for about 90 days. In one embodiment, the contraceptive is released in vitro at a rate of about 35 μg per day to about 70 μg per day for about 30 days. In another embodiment, the contraceptive is released in vitro at a rate of about 35 μg per day to about 70 μg per day for about 60 days. In another embodiment, the contraceptive is released in vitro at a rate of about 35 μg per day to about 70 μg per day for about 90 days.

In one embodiment, the contraceptive is released in vitro at a rate of less than about 100 μg per day for about 30 days. In another embodiment, the contraceptive is released in vitro at a rate of less than about 100 μg per day for about 60 days. In another embodiment, the contraceptive is released in vitro at a rate of less than about 100 μg per day for about 90 days. In one embodiment, the contraceptive is released in vitro at a rate of less than about 70 μg per day for about 30 days. In another embodiment, the contraceptive is released in vitro at a rate of less than about 70 μg per day for about 60 days. In another embodiment, the contraceptive is released in vitro at a rate of less than about 70 μg per day for about 90 days. In one embodiment, the contraceptive is released in vitro at a rate of less than about 35 μg per day for about 30 days. In another embodiment, the contraceptive is released in vitro at a rate of less than about 35 μg per day for about 60 days. In another embodiment, the contraceptive is released in vitro at a rate of less than about 35 μg per day for about 90 days. In one embodiment, the contraceptive is released in vitro at a rate of less than about 15 μg per day for about 30 days. In another embodiment, the contraceptive is released in vitro at a rate of less than about 15 μg per day for about 60 days. In another embodiment, the contraceptive is released in vitro at a rate of less than about 15 μg per day for about 90 days.

Values and ranges included and/or intermediate within the ranges set forth herein are also intended to be within the scope of the present disclosure. Ranges having values recited herein as an upper or lower limit are also intended to be within the scope of the present disclosure.

### IV. Antimicrobial Compounds

The instant invention further provides platinum-catalyzed silicone intravaginal drug delivery devices, e.g., intravaginal rings, comprising a contraceptive having a terminal alkene, alkyne or carbonyl group, and an antimicrobial agent.

As used herein, the term “antimicrobial compound” or “antimicrobial agent” (used interchangeably herein) refers to a compound or agent which is capable of inhibiting or destroying the growth of a microbial organism. In one embodiment, the antimicrobial compound is dapivirine. In one embodiment, the antimicrobial compound is a non-nucleoside reverse transcriptase inhibitor (“NNRTI”). In one embodiment, the antimicrobial compound is a viral entry inhibitor. In one embodiment of the invention, the antimicrobial compound is maraviroc. In one embodiment of the invention, the antimicrobial is DS003. In another embodiment of the invention, the antimicrobial compound is darunavir, GSK 1265744 or BMS-663068. The term “antimicrobial compound” is intended to embrace antibacterial agents, antifungal agents, antiprotozoal agents, antiviral agents and mixtures thereof.

In another embodiment of the invention, the antimicrobial compound is a non-nucleoside reverse transcriptase inhibitor (“NNRTI”). In one embodiment, the NNRTI is a substituted di-amino pyrimidine derivative. Useful NNRTI class compounds include, but are not limited to, nevirapine, delavirdine, etravirine and efavirenz. NNRTIs bind to the hydrophobic pocket near the active site of the HIV reverse transcriptase (RT) enzyme, blocking DNA polymerization. (See, e.g., Tarby, Curr. Top. Med. Chem., 2004; 4(10):1045-57, U.S. Patent Application Publication No. US2006/0166943, and PCT Publication No. WO03/094920, the entire contents of each of which are expressly incorporated herein by reference.) This prevents viral replication and, therefore, production of infectious virus. (Borkow et al., J. Virol., 1997; 71(4):3023-30.)

In another preferred embodiment of the invention, the antimicrobial compound is a viral entry inhibitor. In another embodiment, the viral entry inhibitor is maraviroc. In another preferred embodiment of the invention, a nucleoside reverse transcriptase inhibitor is used.

The term “antimicrobial compound” is intended to embrace antibacterial agents, antifungal agents, antiprotozoal agents, antiviral agents and mixtures thereof.

Suitable antibacterial agents include Acrosoxacin, Amifloxacin, Amoxycillin, Ampicillin, Aspoxicillin, Azidocillin, Azithromycin, Aztreonam, Balofloxacin, Benzylpenicillin, Biapenem, Brodimoprim, Cefaclor, Cefadroxil, Cefatrizine, Cefcapene, Cefdinir, Cefetamet, Cefmetazole, Cefprozil, Cefroxadine, Ceftibuten, Cefuroxime, Cephalexin, Cephalonium, Cephaloridine, Cephamandole, Cephazolin, Cephradine, Chlorquinaldol, Chlortetracycline, Ciclacillin, Cinoxacin, Ciprofloxacin, Clarithromycin, Clavulanic Acid, Clindamycin, Clofazimine, Cloxacillin, Danofloxacin, Dapsone, Demeclocycline, Dicloxacillin, Difloxacin, Doxycycline, Enoxacin, Enrofloxacin, Erythromycin, Fleroxacin, Flomoxef, Flucloxacillin, Flumequine, Fosfomycin, Isoniazid, Levofloxacin, Mandelic Acid, Mecillinam, Metronidazole, Minocycline, Mupirocin, Nadifloxacin, Nalidixic Acid, Nifuirtoinol, Nitrofurantoin, Nitroxoline, Norfloxacin, Ofloxacin, Oxytetracycline, Panipenem, Pefloxacin, Phenoxymethylpenicillin, Pipemidic Acid, Piromidic Acid, Pivampicillin, Pivmecillinam, Prulifloxacin, Rufloxacin, Sparfloxacin, Sulbactam, Sulfabenzamide, Sulfacytine, Sulfametopyrazine, Sulphacetamide, Sulphadiazine, Sulphadimidine, Sulphamethizole, Sulphamethoxazole, Sulphanilamide, Sulphasomidine, Sulphathiazole, Temafloxacin, Tetracycline, Tetroxoprim, Tinidazole, Tosufloxacin, Trimethoprim and salts or esters thereof.

Preferred antibacterial agents include tetracyclines such as Doxycycline, Tetracycline or Minocycline; macrolides such as Azithromycin, Clarithromycin and Erythromycin; nitroimidazoles such as Metronidazole or Tinidazole; quinolones such as Ofloxacin, Norfloxacin, Cinoxacin, Ciprofloxacin and Levofloxacin; Clindamycin and Dapsone.

Suitable antifungal agents include Bifonazole, Butoconazole, Chlordantoin, Chlorphenesin, Ciclopirox Olamine, Clotrimazole, Eberconazole, Econazole, Fluconazole, Flutrimazole, Isoconazole, Itraconazole, Ketoconazole, Miconazole, Nifuroxime, Tioconazole, Terconazole, Undecenoic Acid and salts or esters thereof. In one embodiment, the antifungal agent is ketoconazole.

Preferred antifungal agents include Clotrimazole, Econazole, Fluconazole, Itraconazole, Ketoconazole, Miconazole, Terconazole and Tioconazole.

Suitable antiprotozoal agents include Acetarsol, Azanidazole, Chloroquine, Metronidazole, Nifuratel, Nimorazole, Omidazole, Propenidazole, Secnidazole, Sineflngin, Tenonitrozole, Temidazole, Tinidazole and salts or esters thereof.

Metronidazole, Tinidazole and Chloroquine are most preferred antiprotozoal agents.

Suitable antiviral agents include Acyclovir, Brivudine, Cidofovir, Curcumin, Dapirivine, Desciclovir, 1-Docosanol, Edoxudine, Fameyclovir, Fiacitabine, Ibacitabine, Imiquimod, Lamivudine, Penciclovir, Valacyclovir, Valganciclovir and salts or esters thereof. Curcumin, Acyclovir, Famcyclovir, Dapirivine and Valacyclovir are preferred antiviral agents.

The antiviral agents darunivir, atazanavir, ritonavir, emtricitabine, zidovudine, maraviroc, lopinavir, lamivudine, and fosamprenavir all have a terminal carbonyl bond. In one embodiment, the antiviral agent is Darunivir. In one embodiment, the antiviral agent is atazanavir. In one embodiment, the antiviral agent is ritonavir. In one embodiment, the antiviral agent is emtricitabine. In one embodiment, the antiviral agent is zidovudine. In one embodiment, the antiviral agent is lopinavir. In one embodiment, the antiviral agent is lamivudine. In one embodiment, the antiviral agent is fosamprenavir.

The most preferred antimicrobial agents of this invention include, without limitation, Dapirivine, Metronidazole, Acyclovir, Clotrimazole, Fluconazole, Terconazole, Azithromycin, Erythromycin, Doxycycline, Tetracycline, Minocycline, Clindamycin, Famcyclovir, Valacyclovir, Clarithromycin, a prodrug or salt thereof and combinations thereof.

Mixtures of antibacterial agents, mixtures of antifungal agents; mixtures of antiviral agents; mixtures of antiprotozoal agents and mixtures of agents from two or more of these categories are also envisaged by the present invention. In addition, it is also envisaged that the present invention embraces at least one antimicrobial agent (microstatic and/or microcidal agent) with one or more other pharmaceutically active agent.

In one embodiment of the invention, the intravaginal ring comprises dapivirine and one antimicrobial agent. In another embodiment of the invention, the intravaginal ring comprises dapivirine and at least two, at least three, at least four, or at least five antimicrobial agents. In another embodiment of the invention, the intravaginal ring comprises dapivirine, a antimicrobial agent, and a contraceptive.

Antimicrobial compounds contained in the rings of the present invention are further described at least in U.S. Patent Application Publication Nos. 2012/0093911 and 2006/0166943 and PCT Publication Nos. WO99/50250, WO02/076426 and WO03/094920, the entire contents of each of which are expressly incorporated herein by reference. The antimicrobial compounds contained in the rings of the present invention can be prepared according to art-known procedures. In particular, they are prepared according to the procedures described in EP 1002795, WO 99/50250, WO 99/50256 and WO 00/27828, the entire contents of each of which are incorporated herein by reference.

The antimicrobial compounds contained in the rings of the present invention may have microbicidal activity and have the ability to prevent the transmission of HIV. In particular, they can prevent sexual or vaginal transmission of HIV by preventing either the production of infectious viral particles or infection of uninfected cells. If infected cells in sperm can reach the mucosa, the compounds of the present invention can prevent HIV infection of host cells, such as macrophages, lymphocytes, Langerhans and M cells. Thus, these compounds prevent systemic HIV infection of a human being, exhibiting a prophylactic action against HIV.

In one embodiment, about 10 to about 30 mg of antimicrobial agent is present in the ring. In another embodiment, about 20 mg to about 30 mg of antimicrobial agent is present in the ring. In yet another embodiment, about 10 to about 800 mg, about 50 mg to about 750 mg, about 100 mg to about 700 mg, or about 200 mg to about 600 mg, about 300 mg to about 400 mg, or about 100 mg to about 1600 mg of antimicrobial agent is present in the ring.

In another embodiment, about 15 mg, 16 mg, 25 mg, 32 mg, 100 mg, 150 mg or 250 mg of antimicrobial agent is present in the ring. In another embodiment, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 55 mg, about 60 mg, about 65 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 350 mg, about 400 mg, about 450 mg, about 500 mg, about 550 mg, about 600 mg, about 650 mg, about 700 mg, about 750 mg, about 800 mg, about 900 mg, about 1000 mg, about 1100 mg, about 1200 mg, about 1300 mg, about 1400 mg, about 1500 mg, or about 1600 mg of antimicrobial agent is present in the ring.

In one embodiment, less than about 2 mg of the antimicrobial compound is released from the ring in vitro during an initial 24 hour period of release. In another embodiment, less than about 1 mg of the antimicrobial compound is released from the ring in vitro during an initial 24 hour period of release.

In one embodiment, the antimicrobial compound is released in vitro at a rate of about 200 μg per day to about 8000 μg per day for about 23 days after an initial 7 day period of release. In another embodiment, the antimicrobial compound is released in vitro at a rate of about 200 μg per day to about 8000 μg per day for about 53 days after the initial 7 day period of release. In another embodiment, the antimicrobial compound is released in vitro at a rate of about 200 μg per day to about 8000 μg per day for about 83 days after the initial 7 day period of release. In another embodiment, the antimicrobial compound is released in vitro at a rate of about 200 μg per day to about 2000 μg per day, about 400 μg per day to about 4000 μg per day, about 550 μg per day to about 5500 μg per day, or about 800 μg per day to about 8000 μg per day for about 23 days or about 53 days after the initial 7 day period of release. In another embodiment, the antimicrobial compound is released in vitro at a rate of about 200 μg per day to about 2000 μg per day, about 400 μg per day to about 4000 μg per day, about 550 μg per day to about 5500 μg per day, or about 800 μg per day to about 8000 μg per day for about 83 days after the initial 7 day period of release.

In one embodiment, less than about 8000 μg per day of the antimicrobial compound is released in vitro after the initial 7 day period of release for about 23 days, for about 53 days or for about 83 days. In another embodiment, less than about 5500 μg per day, 4000 μg per day, or 4000 μg per day of the antimicrobial compound is released in vitro after the initial 7 day period of release for about 23 days, for about 53 days, or for about 83 days. In one embodiment, at least about 200 μg per day, 400 μg per day, 550 μg per day, or 800 μg per day of the antimicrobial compound is released in vitro after the initial 7 day period of release for about 23 days, about 53 days, or about 83 days.

In one embodiment, the antimicrobial compound is released in vitro at a rate of about 200 μg per day to about 2000 μg per day for about 30 days, for about 60 days, or for about 90 days. In another embodiment, the antimicrobial compound is released in vitro at a rate of about 400 μg per day to about 4000 μg per day for about 30 days, for about 60 days, or for about 90 days. In one embodiment, the antimicrobial compound is released in vitro at a rate of about 550 μg per day to about 5500 μg per day for about 30 days, for about 60 days, or for about 90 days. In another embodiment, the antimicrobial compound is released in vitro at a rate of about 800 μg per day to about 800 μg per day for about 30 days, for about 60 days, or for about 90 days.

Values and ranges included and/or intermediate within the ranges set forth herein are also intended to be within the scope of the present disclosure. Ranges having values recited herein as an upper or lower limit are also intended to be within the scope of the present disclosure.

Dapivirine

The instant invention provides intravaginal rings comprising a contraceptive and an antimicrobial compound, such as dapivirine. As used herein, the term “dapivirine” refers to (4-[[4-[(2,4,6-trimethylphenyl)amino]-2-pyrimidinyl]amino]benzonitrile), a non-nucleoside reverse transcriptase inhibitor (see structure, below).

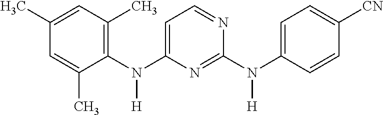 [SMILES: Cc1cc(C)c(Nc2ccnc(Nc3ccc(C#N)cc3)n2)c(C)c1]

Dapivirine is useful in the prevention and/or treatment of retroviral infection, such as HIV-1 infection. It is a crystalline compound that is white to slightly beige in color, has a melting point of about 220° C. and is virtually insoluble in water. More specifically, the solubility of dapivirine is less than 0.001 mg/gm of water (i.e., less than 1 μg/ml of water). The intravaginal rings of the instant invention may use micronized dapivirine. A composite result (four samples taken of micronized material) showed that 88.15% of the material had a particle size of less than 5 microns (μM).

Dapivirine was originally developed as an oral antiretroviral compound and was first conceived as an oral therapeutic. Dapivirine has potent activity against wild-type HIV-1 strains and HIV-1 strains harboring different resistance-inducing mutations. (Das et al., J. Med Chem., 2004; 47(10):2550-60.) Dapivirine is a white to off-white or slightly yellow powder, free from visible impurities, has a melting point of approximately 220° C., and is virtually insoluble in water. Dapivirine, a substituted DAPY derivate with the chemical name 4-[[4-[(2,4,6-trimethylphenyl)amino]-2-pyrimidinyl]amino]benzonitrile, is a non-nucleoside reverse transcriptase inhibitor (NNRTI).

In one embodiment, the invention provides intravaginal rings comprising dapivirine and a contraceptive. In another embodiment, the invention provides intravaginal rings comprising dapivirine, a contraceptive, and an additional antimicrobial agent.

### V. Methods for Preparing Intravaginal Drug Delivery Devices of the Invention

The present invention provides intravaginal drug delivery devices, e.g., intravaginal rings, comprising compounds having terminal alkene, alkyne or carbonyl functionalities. The devices of the invention exhibit increased recovery of the compounds having terminal alkene, alkyne or carbonyl functionalities from platinum-catalyzed silicone polymers due to the optimization of compound particle size and cure conditions used when the device is being prepared. Without intending to be limited by theory, it is believed that by decreasing the amount of the compound having a terminal alkene, alkyne or carbonyl group solubilized in the silicone during the cure cycle, the amount of drug bound to the silicone polymer is reduced to an acceptable level for commercial drug products.

Specifically, the inventors of the instant invention have discovered that the amount of compound having a terminal alkene, alkyne or carbonyl group which is solubilized after mixing into the silicone system is determined by three factors: 1) total surface area of the compound exposed to the silicone polymer; 2) the temperature of the system, and 3) the time the compound has been in contact with the silicone. Thus, compound solubilization can be reduced by using highly crystalline drug material (so that more energy is needed to solubilize the drug), by using larger primary particle size, which reduces the surface area to increase solubilization time, and by reducing thermal exposure. By optimizing platinum-catalyzed silicone intravaginal ring formulations for drug particle size, cure temperature and cure time, the instant invention minimizes the amount of drug which binds to a silicone polymer during the platinum curing process, thereby resulting in higher recovery of the drug from platinum-catalyzed silicone intravaginal drug delivery devices, e.g., intravaginal rings.

Intravaginal drug delivery devices, e.g., intravaginal rings, of the invention may be manufactured by any method known by those skilled-in-the-art, but preferably by injection molding or extrusion, and more preferably by reaction injection molding of silicone elastomer systems. The term “injection molding” refers to manufacturing processes for producing parts/devices from thermosetting materials using suitably designed injection molds. Examples of thermosetting materials include silicone rubbers/elastomers. Without limitation, matrix-type silicone elastomer rings containing a contraceptive may be prepared by (i) adding and mixing the contraceptive into one or more components of the silicone system (e.g., base, crosslinking agent, catalyst, excipient, dispersant, etc.) (ii) injecting the mix into suitably designed injection molds, and (iii) optionally, applying heat to cause the silicone mix to cure/crosslink forming an elastomer.

The present invention provides additional methods of preparing the intravaginal rings of the invention described above. These methods generally comprise dispersing the contraceptive or the contraceptive and an antimicrobial agent, and an elastomer, e.g., polysiloxane, in an appropriate solvent or dispersing agent, e.g., silicone liquid, and curing the rings with a platinum catalyst, e.g., a platinum-siloxane complex, thereby preparing a platinum-catalyzed ring. Any of the well-known elastomers, e.g., polysiloxanes, described supra may be used to prepare the platinum-catalyzed rings of the invention. In one embodiment, an elastomer, e.g., polysiloxane, for use in the methods of the invention is a dimethylsiloxane, e.g., vinyl-terminated polydimethylsiloxane. In another embodiment, an elastomer, e.g., polysiloxane, for use in the methods of the invention is a diorganopopolysiloxane, e.g., dimethylpolysiloxane. In another embodiment, the elastomer, e.g., polysiloxane, for use in the methods of the invention is NuSil MED-8470. In another embodiment, the elastomer for use in the methods of the invention is NuSil DDU-4320. In certain embodiments, the methods further comprise use of a cross-linker, e.g., hydride functional polydimethylsiloxane or dimethylmethylhydrogen polysiloxane cross-link.

In one embodiment, the method further comprises catalyzing the rings in a ring mould. The mould can then be opened, following which the intravaginal ring is removed and trimmed.

Ring moulds, are preferably coated with, for example, Teflon™ or an electrolytically applied metalised coating. Ring moulds may be constructed of hardened carbon steel, stainless steel, aluminum, or any other material deemed to be appropriate. It will be appreciated that the mould dimensions and design impart the physical shape of the intravaginal drug delivery device, for example, a partial or complete ring, or any other desired shape. Preferably, the device has a partial or complete toroidal shape, more preferably a partial or complete torus shape, or a substantially cylindrical shape. By toroid is meant a ring-like body generated by rotating any closed loop (including an ellipse, a circle or any irregular curve) about a fixed line external to that loop. The toroid shape may be a complete or partial toroid. By torus is meant a ring-like body generated by rotating a circle about a fixed line external to the circle. The torus shape may be a complete or partial ring-like shape. The geometric characteristics of the mould and intravaginal rings can be varied as required by the use.

Alternatively, the intravaginal ring device, or components thereof, may be prepared by extrusional processes, e.g., co-extrusion or blend extrusion, well known to those skilled in the art (see, e.g., U.S. Pat. No. 5,059,363, the entire contents of which are incorporated herein by reference). Rings may also be fabricated by extrusion instead of injection molding, whereby the mixture is extruded as a straight rod, cured, and then formed into a ring.

In a specific embodiment of the invention, an intravaginal drug delivery device, e.g., intravaginal ring, of the instant invention can be prepared by creating premixes of appropriate quantities of each active pharmaceutical ingredient (i.e., compound comprising a terminal alkene, alkyne or carbonyl group) with Part A or Part B of a silicone elastomer. For each formulation, Premix A and Premix B can be prepared in a predetermined gram batch size. As a non-limiting example, specific steps for the preparation for a NuSil MED-4870 silicone intravaginal ring premix are as follows:

1. Quantity of API (i.e., compound comprising a terminal alkene, alkyne or carbonyl group), alone or in combination with an antimicrobial compound, such as dapivirine, weighed into SpeedMixer tub.

2. Quantity NuSil MED-4870 (Part A or Part B) added to SpeedMixer tub.

3. SpeedMixer tub sealed and contents mixed using SpeedMixer (3 minutes at 3000 rpm).

4. Premixes stored in refrigerator until required for injection moulding.

5. Remove premix from refrigerator, hand mix for 30 seconds, and then SpeedMix for 120 seconds at 3000 rpm.

Prior to injection moulding of rings, premixes of A and B are combined in a 1:1 ratio according to the following procedure:

1. 50 g of Premix A and 50 g of Premix B are added to SpeedMixer tub in layers (25 g Premix A, then 25 g Premix B, then 25 g Premix A, then 25 g Premix B) and hand mixed for 30 seconds.

2. SpeedMixer tub sealed and contents mixed using SpeedMixer (30 seconds at 3000 rpm).

3. Steps 1 and 2 are repeated for each formulation until 4×100 g total A/B mixture is produced.

The contents of each tub are then transferred to a 500 g cartridge that operates with the dosing system of a Babyplast injection molder. Heating of the ring mold assembly on the Babyplast machine is performed via 2×200 W heater cartridges on both the fixed and mobile plates. Injection parameters are as follows: 100 bar clamping pressure, 50 bar injection pressure, 60° C. to 200° C. mould temperature, 60 seconds to 120 minute cure time. Manufactured rings are then weighed and packaged as required. In an additional embodiment of the invention, MED-360 oil incorporation provides an improvement in recovery of the compound by a slowing of the dissolution rate.

As discussed in detail herein, the amount of drug having a terminal alkene, alkyne or carbonyl group which is solubilized after mixing into the silicone system is determined by three factors: 1) total surface area of the drug exposed to the silicone polymer; 2) the temperature of the system (higher temperature typically equals higher solubility of the drug in the system), and 3) the time the drug has been in contact with the silicone, as reaching equilibrium solubility is a time-dependent process. Examples 4 and 5, below, provide numerous examples of cure times and temperatures in combination with drug particle size.

In one embodiment of the invention, the cure temperature is 60° C. to 200° C. In another embodiment, the cure temperature is 60° C. to 150° C. In another embodiment, the cure temperature is 60° C. to 120° C. In another embodiment, the cure temperature is 60° C. to 100° C. In another embodiment, the cure temperature is 80° C. to 200° C. In another embodiment, the cure temperature is 100° C. to 200° C. In another embodiment, the cure temperature is 120° C. to 200° C. In another embodiment, the cure temperature is 80° C. to 120° C. In another embodiment, the cure temperature is 80° C. to 100° C.

In one embodiment, the cure temperature is about 60° C. In another embodiment, the cure temperature is about 70° C. In another embodiment, the cure temperature is about 80° C. In another embodiment, the cure temperature is about 90° C. In another embodiment, the cure temperature is about 100° C. In another embodiment, the cure temperature is about 110° C. In another embodiment, the cure temperature is about 120° C. In another embodiment, the cure temperature is about 130° C. In another embodiment, the cure temperature is about 140° C. In another embodiment, the cure temperature is about 150° C. In another embodiment, the cure temperature is about 160° C. In another embodiment, the cure temperature is about 170° C. In another embodiment, the cure temperature is about 180° C. In another embodiment, the cure temperature is about 190° C. In another embodiment, the cure temperature is about 200° C. In another embodiment, the cure temperature is about 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., 140° C., 141° C., 142° C., 143° C., 144° C., 145° C., 146° C., 147° C., 148° C., 149° C., 150° C., 151° C., 152° C., 153° C., 154° C., 155° C., 156° C., 157° C., 158° C., 159° C. or 160° C.

In another embodiment of the invention, the cure time is about 80 seconds. In another embodiment of the invention, the cure time is about 90 seconds. In one embodiment of the invention, the cure time is about 60 seconds, about 70 seconds, about 80 seconds, about 90 seconds, about 100 seconds, about 120 seconds, about 140 seconds, about 160 seconds, about 180 seconds, about 200 seconds, about 250 seconds, about 300 seconds, about 350 seconds, about 360 seconds, about 400 seconds, about 450 seconds, about 500 seconds, about 550 seconds, about 600 seconds, or about 650 seconds. In one embodiment of the invention, the cure time is about 60 seconds. In another embodiment of the invention, the cure time is about 70 seconds. In one embodiment of the invention, the cure time is about 3 minutes. In another embodiment of the invention, the cure time is about 4 minutes. In another embodiment of the invention, the cure time is about 5 minutes. In another embodiment of the invention, the cure time is about 6 minutes. In another embodiment of the invention, the cure time is about 7 minutes. In another embodiment of the invention, the cure time is about 8 minutes. In another embodiment of the invention, the cure time is about 9 minutes. In another embodiment of the invention, the cure time is about 10 minutes. In another embodiment of the invention, the cure time is about 15 minutes. In another embodiment of the invention, the cure time is about 20 minutes. In another embodiment of the invention, the cure time is about 25 minutes. In another embodiment of the invention, the cure time is about 30 minutes. In another embodiment of the invention, the cure time is about 60 minutes. In another embodiment of the invention, the cure time is about 90 minutes.

Values and ranges included and/or intermediate within the ranges set forth herein are also intended to be within the scope of the present disclosure. Ranges having values recited herein as an upper or lower limit are also intended to be within the scope of the present disclosure.

### VI. Methods for Assaying Recovery or Covalent Binding of Compounds Having a Terminal Alkene, Alkyne or Carbonyl Group to Platinum-Catalyzed Silicone Polymer Drug Delivery Devices

Methods for assaying the percent of compound having a terminal alkene, alkyne or carbonyl group recoverable from or covalently bound to a platinum-catalyzed silicone intravaginal drug delivery device of the invention are commonly known in the art. For example, silicone intravaginal rings, slabs or IUDs can be added to a glass flask. 5 mL of a 2.5 mg/mL norethindrone solution (NOR; present as an internal standard) in methanol can be added to the flask along with 95 mL of dichlormethane (DCM). The flask can be placed in a rotating orbital incubator set at 37° C. and 60 rpm for 72 hours. A 2 mL aliquot of the cooled extraction mixture can be transferred to a glass centrifuge tube and allowed to evaporate to dryness. The dried samples can be reconstituted in 10 mL of methanol with vortex mixing for 30 seconds and sonication for 40 minutes. A 1:10 dilution of this solution can be prepared using methanol and water such that the final solvent composition is 1:1. This solution and be analyzed by HPLC. Recovery can be expressed as a percentage of predicted compound as compared to the original amount of compound present in the silicone intravaginal ring, slab or IUD. The final data can be corrected based on the recovery of a control solution of known compound concentration that was “extracted” at the same time.

Other methods for assaying the percent of compound having a terminal alkene, alkyne or carbonyl group recoverable from or covalently bound to a platinum-catalyzed silicone intravaginal drug delivery device of the invention include HPLC-MS or GC-MS. Other solvents besides those listed above may be used to extract the active pharmaceutical ingredient from the ring, including but not limited to, acetone, acetonitrile or hexane.

To determine the amount of compound having a terminal alkene, alkyne or carbonyl group that is covalently bound to the platinum-catalyzed silicone intravaginal drug delivery device, the percentage of compound recovered from the device can be subtracted from the original amount of compound present in the device.

Alternatively, nuclear magnetic resonance (NMR) experiments can be performed to investigate the binding of compound having a terminal alkene, alkyne or carbonyl group to the silicone elastomer polymer drug delivery device of the invention. For example, silicone intravaginal rings, slabs or IUDs can be dissolved or swollen in deuterated chloroform (CDCl₃) prior to analysis. For solution-state NMR, ¹H and ¹³C NMR spectra can be measured on a spectrometer, and spectra can be recorded with a variable number of scans. Chemical shifts can be recorded in parts per million, with the chemical shift of the internal reference set to 77.0 ppm for ¹³C spectra and 7.26 ppm for ¹H spectra with respect to the CDCl₃ solvent. For solid-state NMR, ¹H and ¹³C and ²⁹Si spectra can be measured on a Varian VNMRS with a 9.4T magnet (operating at 399.88 MHz for ¹H). Spectra can be obtained using a variable number of scans.

In other embodiments, Raman or IR (infrared) could be used to investigate the binding of the compound having a terminal alkene, alkyne or carbonyl group to the silicone elastomer polymer drug delivery device of the invention.

### VII. Methods for Determining Primary Particle Size and Particle Size Distribution of Compounds Having Terminal Alkene, Alkynes and/or Carbonyl Groups

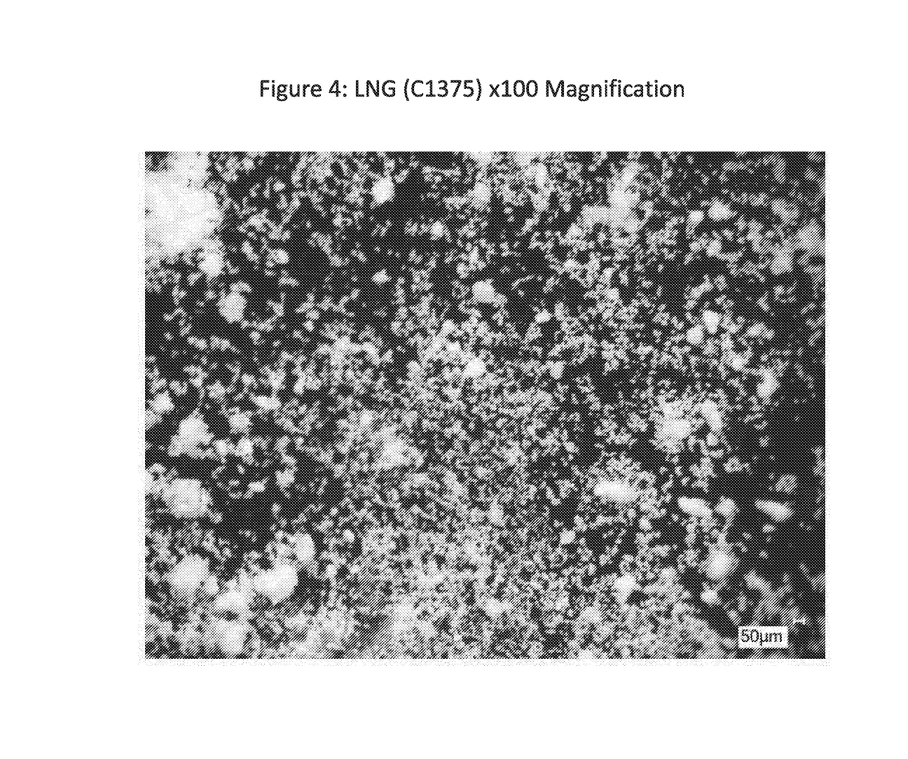

Methods for determining the primary particle size and particle size distribution of a compound having a terminal alkene, alkyne or carbonyl group are commonly known in the art. For example, digital microscopy can be performed on the batch of the compound to determine the primary particle size. As a non-limiting example, a small sample of the active pharmaceutical ingredient (i.e., compound comprising a terminal alkene, alkyne or carbonyl group) can be dusted on a section of adhesive tape to provide a thin layer of API, and dilute suspensions of the API can be prepared in MED-360 silicone fluid for particle shape and size analysis using digital microscopy at a range of magnifications. Example 3, below, and FIGS. 4-7 provide examples of such digital microscopy techniques.

As used herein, the term “primary particle size” refers to the average size of the smallest particle of a compound having a terminal alkene, alkyne or carbonyl group of the invention. Primary particle size is often defined by the d50, diameter at which 50% of the sample's measured particles are smaller sized particles, or by the d90, diameter at which 90% of the sample's measured particles are smaller sized particles. d50 and d90 values are commonly determined by one of ordinary skill in the art using various techniques, such as laser diffraction, dynamic light scattering, electrophoretic light scattering, automated imaging, sedimentation, electrozone sensing, light obscuration, image analysis, and sieving. Primary particles often agglomerate into larger structures comprising multiple primary particles (see, for example FIG. 8(A)).

In one embodiment of the invention, the compound comprising a terminal alkene, alkyne or carbonyl group has a d50 of greater than or equal to about 40 microns. In another embodiment, the compound comprising a terminal alkene, alkyne or carbonyl group has a d50 of greater than or equal to about 50 microns. In another embodiment, the compound comprising a terminal alkene, alkyne or carbonyl group has a d50 of greater than or equal to about 60 microns. In another embodiment, the compound comprising a terminal alkene, alkyne or carbonyl group has a d50 of greater than or equal to about 70 microns. In another embodiment, the compound comprising a terminal alkene, alkyne or carbonyl group has a d50 of greater than or equal to about 80 microns. In another embodiment, the compound comprising a terminal alkene, alkyne or carbonyl group has a d50 of greater than or equal to about 90 microns. In another embodiment, the compound comprising a terminal alkene, alkyne or carbonyl group has a d50 of greater than or equal to about 100 microns. In another embodiment, the compound comprising a terminal alkene, alkyne or carbonyl group has a d50 of greater than or equal to about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 80, 81, 82, 83, 84, 85, 86, 87, 88, 89, 90, 91, 92, 93, 94, 95, 96, 97, 98, 99 or 100 microns.

In another embodiment of the invention, the compound comprising a terminal alkene, alkyne or carbonyl group has a d50 of about 40 microns to about 500 microns. In another embodiment of the invention, the compound comprising a terminal alkene, alkyne or carbonyl group has a d50 of about 40 microns to about 400 microns. In another embodiment of the invention, the compound comprising a terminal alkene, alkyne or carbonyl group has a d50 of about 40 microns to about 300 microns. In another embodiment of the invention, the compound comprising a terminal alkene, alkyne or carbonyl group has a d50 of about 40 microns to about 200 microns. In another embodiment of the invention, the compound comprising a terminal alkene, alkyne or carbonyl group has a d50 of about 40 microns to about 150 microns. In another embodiment of the invention, the compound comprising a terminal alkene, alkyne or carbonyl group has a d50 of about 40 microns to about 125 microns. In another embodiment of the invention, the compound comprising a terminal alkene, alkyne or carbonyl group has a d50 of about 40 microns to about 100 microns. In another embodiment of the invention, the compound comprising a terminal alkene, alkyne or carbonyl group has a d50 of about 40 microns to about 80 microns. In another embodiment of the invention, the compound comprising a terminal alkene, alkyne or carbonyl group has a d50 of about 40 microns to about 60 microns. In another embodiment of the invention, the compound comprising a terminal alkene, alkyne or carbonyl group has a d50 of about 100 microns to about 500 microns. In another embodiment of the invention, the compound comprising a terminal alkene, alkyne or carbonyl group has a d50 of about 200 microns to about 500 microns. In another embodiment of the invention, the compound comprising a terminal alkene, alkyne or carbonyl group has a d50 of about 60 microns to about 200 microns. In another embodiment of the invention, the compound comprising a terminal alkene, alkyne or carbonyl group has a d50 of about 50 microns to about 80 microns.

In another embodiment of the invention, a compound comprising a terminal alkene, alkyne or carbonyl group has a d90 of about 80 microns. In another embodiment of the invention, a compound comprising a terminal alkene, alkyne or carbonyl group has a d90 of about 90 microns. In another embodiment of the invention, a compound comprising a terminal alkene, alkyne or carbonyl group has a d90 of about 100 microns. In another embodiment of the invention, a compound comprising a terminal alkene, alkyne or carbonyl group has a d90 of about 100 microns. In another embodiment of the invention, a compound comprising a terminal alkene, alkyne or carbonyl group has a d90 of about 120 microns. In another embodiment of the invention, a compound comprising a terminal alkene, alkyne or carbonyl group has a d90 of about 130 microns. In another embodiment of the invention, a compound comprising a terminal alkene, alkyne or carbonyl group has a d90 of about 135 microns. In another embodiment of the invention, a compound comprising a terminal alkene, alkyne or carbonyl group has a d90 of about 140 microns. In another embodiment of the invention, a compound comprising a terminal alkene, alkyne or carbonyl group has a d90 of about 150 microns. In another embodiment of the invention, a compound comprising a terminal alkene, alkyne or carbonyl group has a d90 of about 160 microns. In another embodiment of the invention, a compound comprising a terminal alkene, alkyne or carbonyl group has a d90 of about 170 microns. In another embodiment of the invention, a compound comprising a terminal alkene, alkyne or carbonyl group has a d90 of about 180 microns. In another embodiment of the invention, a compound comprising a terminal alkene, alkyne or carbonyl group has a d90 of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159 or 160 microns.

In another embodiment of the invention, a compound comprising a terminal alkene, alkyne or carbonyl group has a d90 of about 80 microns to about 500 microns. In another embodiment, a compound comprising a terminal alkene, alkyne or carbonyl group has a d90 of about 80 microns to about 400 microns. In another embodiment, a compound comprising a terminal alkene, alkyne or carbonyl group has a d90 of about 80 microns to about 300 microns. In another embodiment, a compound comprising a terminal alkene, alkyne or carbonyl group has a d90 of about 80 microns to about 250 microns. In another embodiment, a compound comprising a terminal alkene, alkyne or carbonyl group has a d90 of about 80 microns to about 200 microns. In another embodiment, a compound comprising a terminal alkene, alkyne or carbonyl group has a d90 of about 80 microns to about 150 microns. In another embodiment, a compound comprising a terminal alkene, alkyne or carbonyl group has a d90 of about 80 microns to about 100 microns.

Values and ranges included and/or intermediate within the ranges set forth herein are also intended to be within the scope of the present disclosure. Ranges having values recited herein as an upper or lower limit are also intended to be within the scope of the present disclosure.

### VIII. Methods for Preventing/Treating Pregnancy and/or HIV

The present invention provides methods preventing pregnancy using the intravaginal rings and devices of the invention comprising a contraceptive, such as levonorgestrel, and/or methods of preventing and/or treating HIV using the intravaginal rings and devices of the invention comprising an antimicrobial agent and a contraceptive.

In one aspect, the invention provides methods of preventing pregnancy in a female human, comprising the step of inserting an intravaginal ring comprising a contraceptive of the invention into the vagina of the female human. In another aspect, the present invention provides methods of blocking DNA polymerization by an HIV reverse transcriptase enzyme in a female human, comprising the step of inserting an intravaginal ring of the invention comprising an antimicrobial compound into the vagina of the female human. In another aspect, the present invention provides methods of preventing HIV infection in a female human, comprising the step of inserting an intravaginal ring of the invention into the vagina of the female human. In yet another aspect, the invention provides methods of treating HIV infection in a female human, comprising the step of inserting an intravaginal ring of the invention into the vagina of the female human.

The ring that is inserted into a female human may contain a prophylactically effective amount or a therapeutically effective amount of a contraceptive, e.g., levonorgestrel. Alternatively, the ring that is inserted into a female human may contain a prophylactically effective amount or a therapeutically effective amount of a contraceptive, e.g., levonorgestrel, and a prophylactically effective amount or a therapeutically effective amount of an antimicrobial agent, e.g., dapivirine.

As used herein, the term “prophylactically effective amount” refers to the amount of compound effective to prevent development of a condition or a disease in the subject. In one embodiment of the invention, the condition is conception or pregnancy. In one embodiment of the invention, the disease is HIV.

As used herein, the term “prophylactically effective amount” refers to the amount of contraceptive effective to prevent contraception or pregnancy in the subject. In one embodiment of the invention, a prophylactically effective amount is achieved when less than about 2 g of the contraceptive is released in vitro during an initial 24 hour period of release. In another embodiment, a prophylactically effective amount is achieved when less than about 1 g of the contraceptive is released in vitro during an initial 24 hour period of release. In one embodiment of the invention, a prophylactically effective amount is achieved when about 20 μg per day to about 290 μg per day of the contraceptive is released in vitro for about 23 days after the initial 7 day period of release. In one embodiment of the invention, a prophylactically effective amount is achieved when about 20 μg per day to about 290 μg per day of the contraceptive is released in vitro for about 53 days after the initial 7 day period of release. In one embodiment of the invention, a prophylactically effective amount is achieved when about 20 μg per day to about 290 μg per day of the contraceptive is released in vitro for about 83 days after the initial 7 day period of release. In one embodiment of the invention, a prophylactically effective amount is achieved when about 35 μg per day to about 70 μg per day of the contraceptive is released in vitro for about 23 days after the initial 7 day period of release. In one embodiment of the invention, a prophylactically effective amount is achieved when about 35 μg per day to about 70 μg per day of the contraceptive is released in vitro for about 53 days after the initial 7 day period of release. In one embodiment of the invention, a prophylactically effective amount is achieved when about 35 μg per day to about 70 μg per day of the contraceptive is released in vitro for about 83 days after the initial 7 day period of release.

In one embodiment of the invention, a prophylactically effective amount is achieved when less than about 100 μg per day of the contraceptive is released in vitro after the initial 7 day period of release for about 23 days. In one embodiment of the invention, a prophylactically effective amount is achieved when less than about 100 μg per day of the contraceptive is released in vitro after the initial 7 day period of release for about 53 days or about 83 days. In one embodiment of the invention, a prophylactically effective amount is achieved when less than about 70 μg per day of the contraceptive is released in vitro after the initial 7 day period of release for about 23 days. In one embodiment of the invention, a prophylactically effective amount is achieved when less than about 70 μg per day of the contraceptive is released in vitro after the initial 7 day period of release for about 53 days or about 83 days. In one embodiment of the invention, a prophylactically effective amount is achieved when less than about 15 μg per day of the contraceptive is released in vitro after the initial 7 day period of release for about 23 days. In one embodiment of the invention, a prophylactically effective amount is achieved when less than about 15 μg per day of the contraceptive is released in vitro after the initial 7 day period of release for about 53 days or about 83 days. In one embodiment of the invention, a prophylactically effective amount is achieved when about 35 μg per day of the contraceptive is released in vitro after the initial 7 day period of release for about 23 days or for about 53 days or about 83 days.

In one embodiment of the invention, a prophylactically effective amount is achieved when about 20 μg per day to about 290 μg per day of the contraceptive is released in vitro for about 30 days. In one embodiment of the invention, a prophylactically effective amount is achieved when about 20 μg per day to about 290 μg per day of the contraceptive is released in vitro for about 60 days. In one embodiment of the invention, a prophylactically effective amount is achieved when about 20 μg per day to about 290 μg per day of the contraceptive is released in vitro for about 90 days. In one embodiment of the invention, a prophylactically effective amount is achieved when about 35 μg per day to about 70 μg per day of the contraceptive is released in vitro for about 30 days. In one embodiment of the invention, a prophylactically effective amount is achieved when about 35 μg per day to about 70 μg per day of the contraceptive is released in vitro for about 60 days. In one embodiment of the invention, a prophylactically effective amount is achieved when about 35 μg per day to about 70 μg per day of the contraceptive is released in vitro for about 90 days.

In one embodiment of the invention, a prophylactically effective amount is achieved when less than about 100 μg per day of the contraceptive is released in vitro for about 30 days. In one embodiment of the invention, a prophylactically effective amount is achieved when less than about 100 μg per day of the contraceptive is released in vitro for about 60 days or about 90 days. In one embodiment of the invention, a prophylactically effective amount is achieved when less than about 70 μg per day of the contraceptive is released in vitro for about 30 days. In one embodiment of the invention, a prophylactically effective amount is achieved when less than about 70 μg per day of the contraceptive is released in vitro for about 60 days or about 90 days. In one embodiment of the invention, a prophylactically effective amount is achieved when less than about 35 μg per day of the contraceptive is released in vitro for about 30 days. In one embodiment of the invention, a prophylactically effective amount is achieved when less than about 35 μg per day of the contraceptive is released in vitro for about 60 days or about 90 days. In one embodiment of the invention, a prophylactically effective amount is achieved when less than about 15 μg per day of the contraceptive is released in vitro for about 30 days. In one embodiment of the invention, a prophylactically effective amount is achieved when less than about 15 μg per day of the contraceptive is released in vitro for about 60 days or about 90 days.

Values and ranges included and/or intermediate within the ranges set forth herein are also intended to be within the scope of the present disclosure. Ranges having values recited herein as an upper or lower limit are also intended to be within the scope of the present disclosure.

Those of skill in the prevention and/or treatment of HIV and the prevention of pregnancy could determine the appropriate therapeutically effective amount or prophylactically effective amount from the data presented here in the Examples section. The exact dosage may depend on the particular active agent used.

The term “subject” means female humans who use the intravaginal rings. Administration of the rings of the present invention to a subject can be carried out using known procedures, at dosages and for periods of time effective to treat or prevent HIV or to prevent pregnancy.

As used herein, the term “vagina” or “vaginal” refers to the passage leading from the opening of the vulva to the cervix of the uterus in female humans. As used herein, the term “intravaginal administering” refers to the administration of a ring of the invention to the vagina of a female human.

The rings of the present invention may be administered into the vagina of a subject prior to sexual intercourse, e.g., 1, 2, 3, 4, 5 or 6 weeks, prior to sexual intercourse. In some embodiments, the rings of the invention may be administered into the vagina of a subject after sexual intercourse, e.g., 1, 2, 3, 4, 5, 6, 7, 8, 9, 10, 11, 12, 13, 14 or 15 days after sexual intercourse.

The term sexual intercourse means vaginal sex.

The term “partners” as used herein defines two or more humans, who are sexually active with each other, i.e., who have sexual intercourse with each other.

As used herein, the term “preventing pregnancy” includes the application or administration of an intravaginal ring of the invention to a subject who is at risk of becoming pregnant in order to decrease the likelihood that the subject will become pregnant. In one embodiment of the invention, the term “preventing pregnancy” includes the application or administration of an intravaginal ring of the invention to a subject who is at risk of becoming pregnant in order to decrease the likelihood that the subject will become pregnant, as compared to a subject who has not been administered an intravaginal ring. In one embodiment of the invention, proper use of the intravaginal rings of the invention leads to prevention of pregnancy infection in about 1%, about 5%, about 10%, about 15%, about 20%, about 25%, about 30%, about 35%, about 40%, about 45%, about 50%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% of the subjects who are at risk of becoming pregnant. Values and ranges included and/or intermediate within the ranges set forth herein are also intended to be within the scope of the present disclosure. Ranges having values recited herein as an upper or lower limit are also intended to be within the scope of the present disclosure.

As used herein, the term “preventing HIV infection” or “preventing HIV transmission” includes the application or administration of an intravaginal ring of the invention to a subject who is at risk of developing HIV, or who has been exposed to but not yet developed HIV, in order to decrease the likelihood that the subject will develop HIV. In one embodiment of the invention, the term “preventing HIV infection” includes the application or administration of an intravaginal ring of the invention to a subject who is at risk of developing HIV, or who has been exposed to but not yet developed HIV, in order to decrease the likelihood that the subject will develop HIV, as compared to a subject who has not been administered an intravaginal ring. In one embodiment of the invention, proper use of the intravaginal rings of the invention leads to prevention of HIV infection in about 1%, about 5%, about 10%, about 15%, about 20%, about 25%, about 30%, about 35%, about 40%, about 45%, about 50%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% of the subjects who are at risk of developing HIV or who have been exposed to but not yet developed HIV. Values and ranges included and/or intermediate within the ranges set forth herein are also intended to be within the scope of the present disclosure. Ranges having values recited herein as an upper or lower limit are also intended to be within the scope of the present disclosure.

The term “treating” includes the application or administration of an intravaginal ring of the invention to a subject, or application or administration of an intravaginal ring of the invention to a subject who has HIV, with the purpose of curing, healing, alleviating, relieving, altering, remedying, ameliorating, preventing, improving, or affecting HIV. The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; or improving a subject's physical or mental well-being. Treatment may be therapeutic or prophylactic. The treatment or amelioration of symptoms can be based on objective or subjective parameters; including the results of a physical examination.

The present invention is further illustrated by the following examples, which should not be construed as further limiting. The contents of all figures and all references, patents and published patent applications cited throughout this application, as well as the Figures, are expressly incorporated herein by reference in their entirety.

### Examples

### Example 1: Evidence of Binding of Contraceptive to Silicone

Formulations of dapivirine (DAP or DPV) with a contraceptive hormone were investigated for use with a multipurpose prevention technology (MPT) intravaginal ring system. Dapivirine and non-micronized levonorgestrel (LNG), micronized ethinyl estradiol (EE), or micronized etonogestrel (ET) were studied in condensation cured (Sn) or addition cured (Pt) silicone systems.

Matrix-type vaginal rings were manufactured by injection moulding, using either MEDS-6382 (condensation-cured) or LSR9-9508-30 (addition-cured) silicone elastomers. Release of DAP and LNG from the LSR9-9508-30 rings was diffusion-controlled for the duration of the 21-day study. Total DAP release was 41% of the initial loading, while only 29% LNG loading was released. Both drugs would have continued to be released beyond 21 days had the study had continued. However, release of EE and ET ceased after 13 and 8 days respectively, with only 36 and 23% of initial drug loading released. Total release of both EE and ET was greater from MEDS-6382 than LSR9-9508-30.

Total drug content for each formulation was not tested. However, these studies indicate that non-micronized active pharmaceutical ingredient may be more recoverable from platinum-catalyzed silicones. Additionally, these studies indicate that LNG, and other drugs in its class, may irreversibly bind to platinum-cured silicones but are recoverable from tin-cured silicones.

### Example 2: Release of Micronized Versus Non-Micronized Levonorgestrel

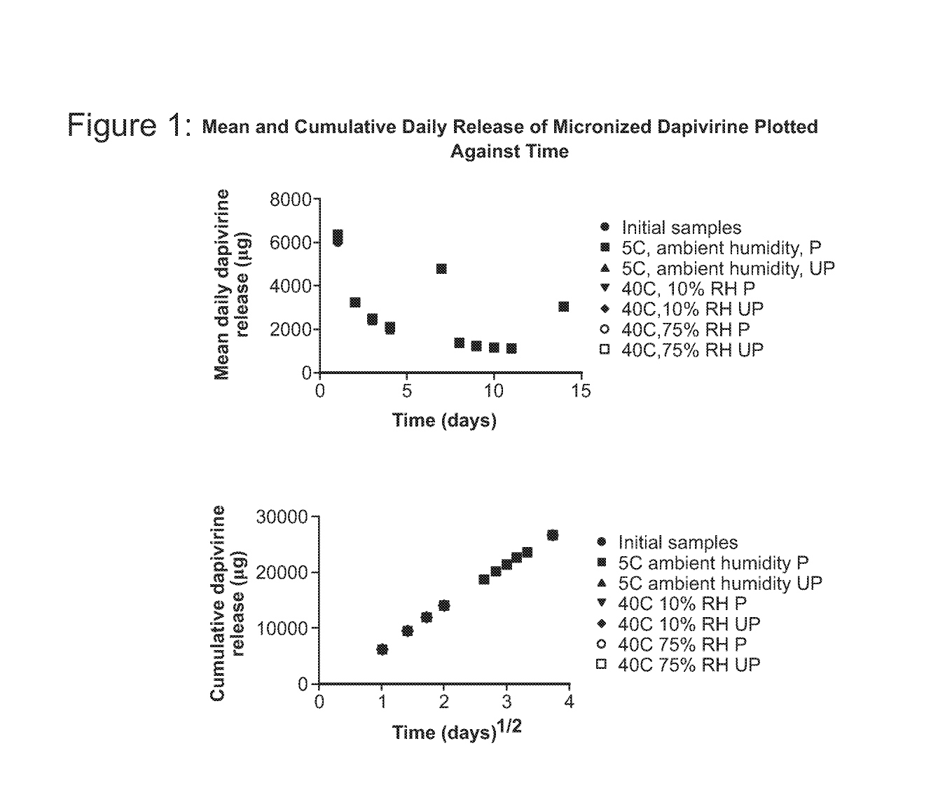

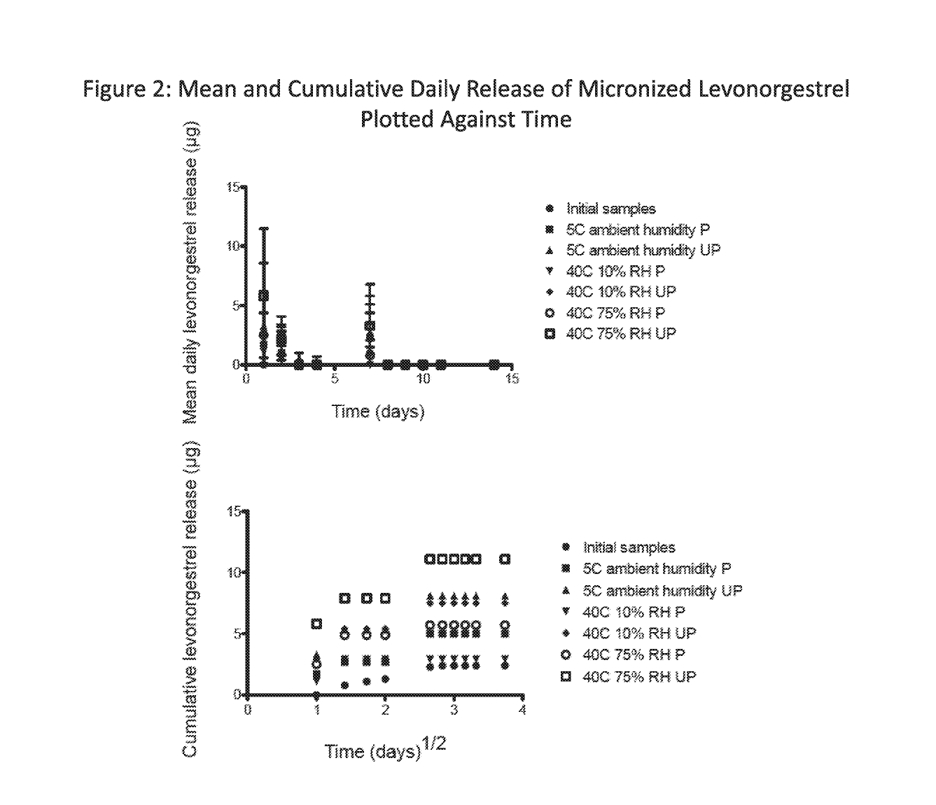

Platinum-catalyzed silicone matrix intravaginal rings comprising 200 mg micronized dapivirine and 32 mg levonorgestrel (both micronized and non-micronized) were utilized in this study. FIGS. 1 and 2 depict the mean and cumulative daily release of dapivirine and levonorgestrel versus time for micronized levonorgestrel and micronized dapivirine on storage stability.

Due to the negligible release of levonorgestrel detected initially (T₀), two of the rings that had been tested were immediately assayed for residual content along with two equivalent rings from the non-micronized levonorgestrel arm of the study. The residual content assay results showed almost no recovery of levonorgestrel from rings containing micronized levonorgestrel and a reduced recovery from those rings containing non-micronized levonorgestrel. Data collected from the week-two time point (see FIGS. 1 and 2) showed a similar lack of levonorgestrel release despite reasonable dapivirine release.

When the week four data showed similar trends initially, the micronized levonorgestrel arm of the study was halted. It was not possible to recover levonorgestrel from rings manufactured with micronized material. This suggests that there is loss of the drug at some point in the manufacturing process. For the non-micronized levonorgestrel rings, there was less than 100% recovery of the amount of levonorgestrel added in two of the rings tested for content after initial release rate testing. This suggests that the loss of levonorgestrel seen with the micronized material also occurs to some extent with the non-micronized material. All of the levonorgestrel release rate results obtained in this study are likely to have been influenced by this phenomenon.

A possible explanation for this observed trend lies in the hypothesized reaction of the ethynl group on the levonorgestrel with available silicone hydride groups on the silicone polymer backbone. As a potentially temperature dependent interaction, this may set up a disequilibrium of dissolved drug within the ring due to the heat distribution throughout the ring during curing. Further work to definitively demonstrate any potential interaction between levonorgestrel and the polymer side chains is described below.

### Example 3: Optimization of Drug Particle Size

Surprisingly, non-micronized levonorgestrel, with larger particle size, appeared to have increased release from the platinum catalyzed silicone rings as compared to the micronized levonorgestrel material, with smaller particle size. Since there seemed to be a difference in release profile from dapivirine/levonorgestrel rings made with non-micronized versus micronized levonorgestrel, formulations were made with levonorgestrel with different particle size distribution (PSD) and from different suppliers.

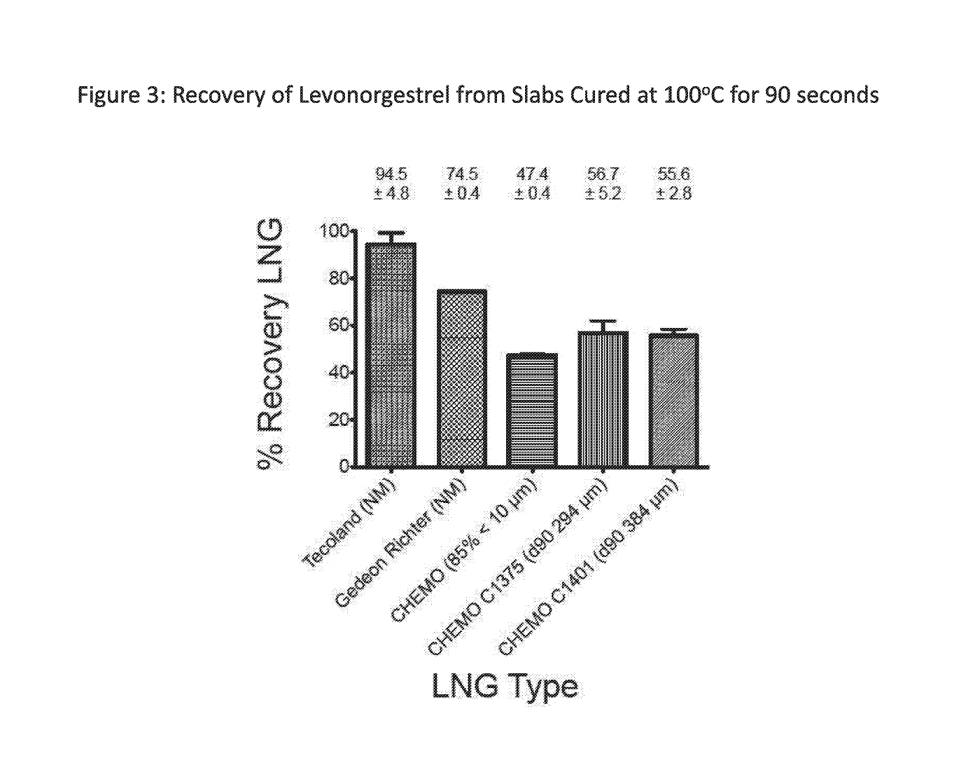

FIG. 3 depicts the recovery of levonorgestrel from formulations made with different batches of levonorgestrel. At the processing conditions shown, recovery non-micronized levonorgestrel supplied by Chemo was significantly lower than recovery of non-micronized LNG supplied by Tecoland.

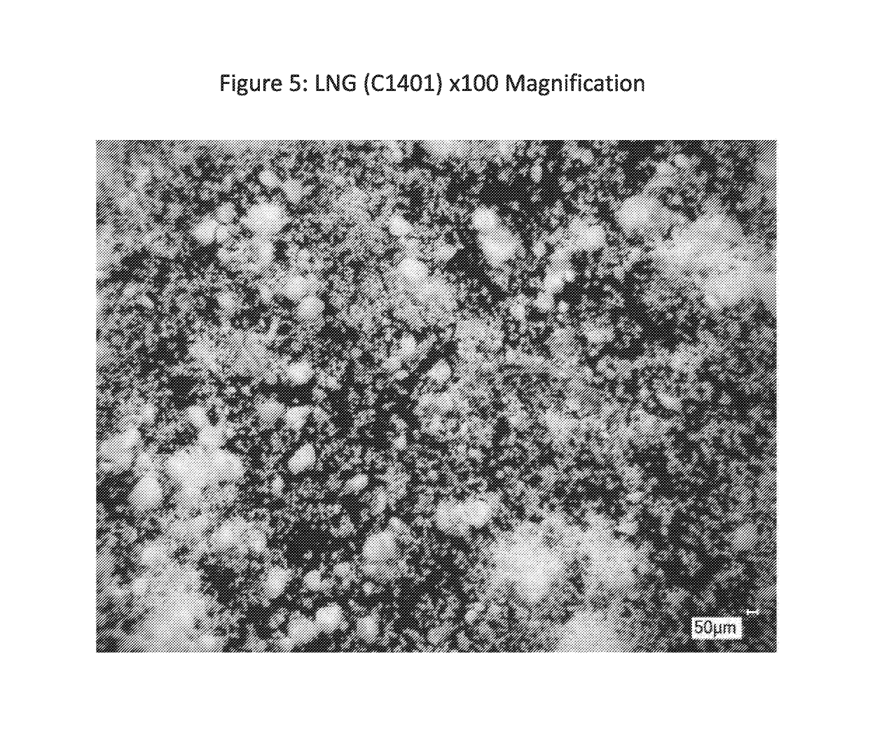

Two additional Chemo LNG batches (Materials 5 and 6, C1375 and C1401) described as having large particle size (d90 values of 294 μm and 384 μm, respectively) were also evaluated. FIGS. 4 and 5 show the microscopy images for LNGC1375 and LNGC1401, respectively. Although described by the supplier as having large PSD, the micrographs clearly show that the bulk properties of the C1401 and C1375 active pharmaceutical ingredients are very similar to those of the micronized LNG API (Material 4). The LNGC1375 and LNGC1401 materials contain a combination of small primary particles and larger physically agglomerated particles. These agglomerates most likely account for the larger particle dimensions quoted by the material supplier. However, it is likely that the smaller particles still present within the agglomerations are similar in size and shape to those of the original micronized material. Furthermore, given the high proportion of small particles present within these materials, the LNGC1375 and LNGC1401 active pharmaceutical ingredients, once incorporated into a silicone elastomer material, behave in a similar manner to the micronized levonorgestrel material.

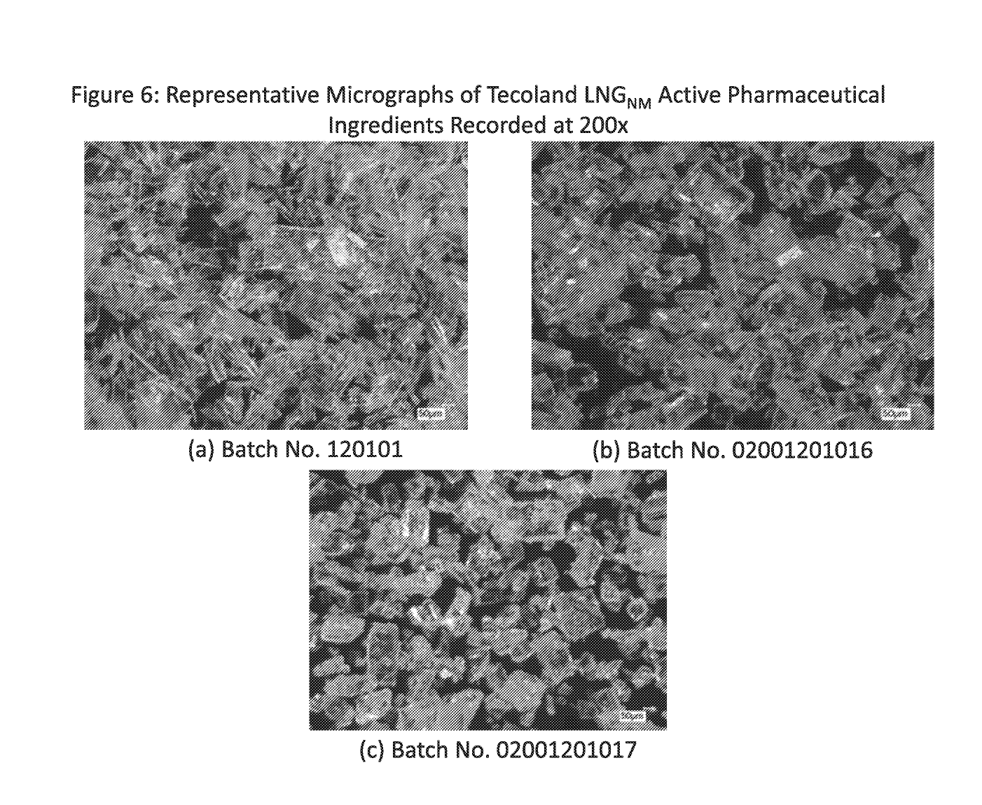

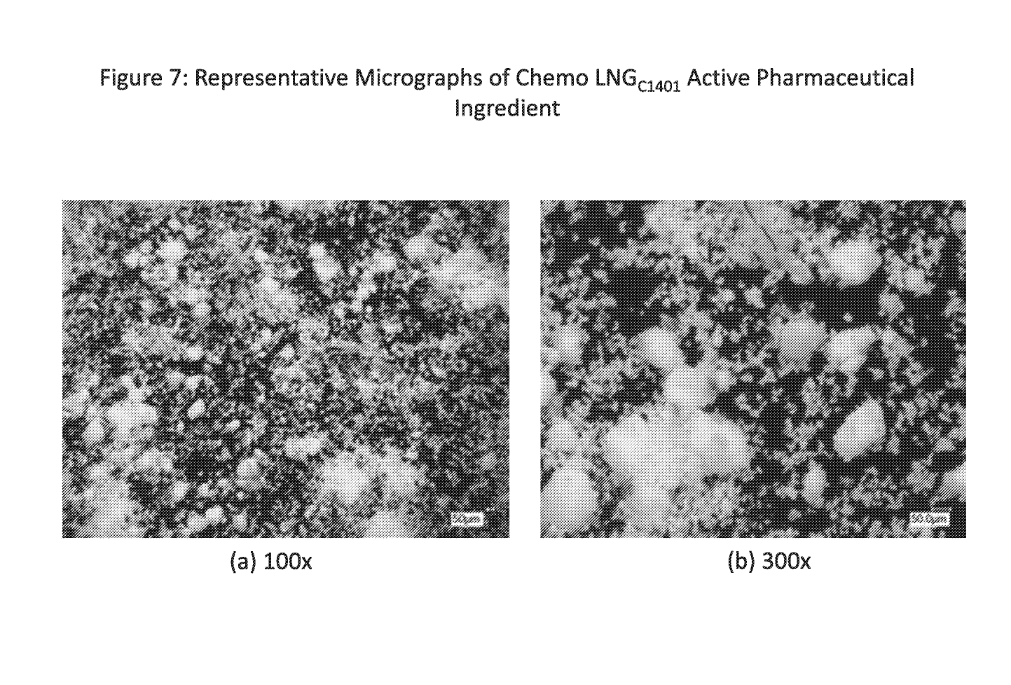

Microscopy images also show that non-micronized levonorgestrel sourced from Tecoland have large primary particles, measuring in the 50-200 micron range (see FIG. 6). Non-micronized levonorgestrel from Chemo is comprised of aggregates of crystals which are also in the 50-200 micron range, but with a much smaller primary particle size (see FIG. 7).

Although these two lots of LNG (Tecoland and Chemo suppliers) have similar particle size distributions and the same PXRD characteristic, they produced formulation samples with different degrees of covalent binding to the silicone polymer. Therefore, large particle size alone is not sufficient to reduce binding of levonorgestrel to silicones. The material must also be highly crystalline and of large primary particle size (d50>90 um). See, for example, FIGS. 8(A) and 8(B).

### Example 4: Optimization of Silicone Polymer Cure Conditions

The influence of silicone polymer system cure time, and cure temperature on levonorgestrel was also investigated. With the understanding that the NuSil DDU-4320 could be cured at a lower temperature than NuSil MED-4870, process optimization proceeded with NuSil DDU-4320.

From cure time and cure temperature experiments, 59% of the added levonorgestrel was recovered from a NuSil DDU-4320 slab heated at 160° C. for 7 minutes. However, a NuSil MED-4870 slab heated at 160° C. for 5 minutes contained only 47% levonorgestrel. Thus, despite a shorter cure time, the NuSil MED-4870 material displayed a lower levonorgestrel content. However, the relative importance of each of these factors was unknown, so it is important to note that the cure temperature and time required, both higher for NuSil MED-4870, will also have a large impact on the amount of levonorgestrel recovered. It is also worth noting that no levonorgestrel was recovered from rings made NuSil MED-4870 using micronized material when these were put on release immediately after manufacture, so all of the levonorgestrel had been reacted off by this stage.

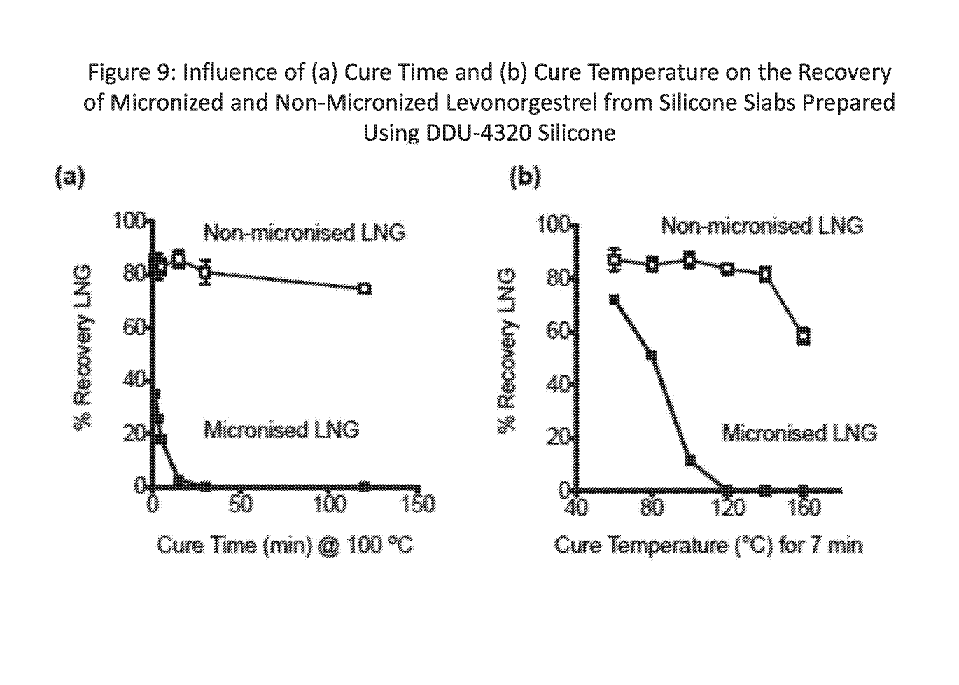

For a given set of cure conditions, non-micronized material showed higher levonorgestrel recovery than micronized material. The recovery of levonorgestrel from NuSil DDU-4320 silicone slabs prepared using varying cure times and temperatures, and incorporating either micronized or non-micronized levonorgestrel (LNG) is displayed in FIGS. 9 (a) and (b). Some LNG loss was observed in all of samples tested. However, loss for micronized LNG was significantly greater than that for non-micronized LNG. For example, at a cure temperature and time of 100° C. for 7 minutes, approximately 87% of the LNG added was recovered using non-micronized LNG, compared with only 12% for micronized LNG. LNG recovery decreased with increasing cure temperature and cure time for both the micronized and non-micronized LNG. For example, the recovery of micronized LNG falls from 35% to zero as the cure time at 100° C. is increased from 1.5 minutes to 30 minutes. Similarly, the recovery of non-micronized LNG falls from approximately 87% to 59% as the cure temperature is increased from 60° C. to 160° C. at a cure time of 7 minutes.

There may also be a plateau effect for the non-micronized LNG, as increasing the cure time at 100° C. from 1.5 minutes to 120 minutes had limited impact, giving recoveries of approximately 84% and 75%, respectively. Similarly, a plateau effect with temperature may also be in effect, as increasing the cure temperature from 60° C. to 140° C. resulted in only a modest reduction in LNG recovered from 87% to 82% with a 7 minute cure time. The results of these experiments are depicted graphically, below.

| | Micronized (M) or | | | |
|---|---|---|---|---|
| % LNG | Non-Micronized | Cure | Cure time | Type of |
| Recoverable | (Non-M) | temp. | (minutes) | silicone |
| 37% | M | 100° C. | 1.5 | DDU-4320 |
| 25% | M | 100° C. | 3 | DDU-4320 |
| 18% | M | 100° C. | 7 | DDU-4320 |
| 2% | M | 100° C. | 15 | DDU-4320 |
| 0% | M | 100° C. | 30 | DDU-4320 |
| 0% | M | 100° C. | 120 | DDU-4320 |
| 81% | Non-M | 100° C. | 1.5 | DDU-4320 |
| 82% | Non-M | 100° C. | 3 | DDU-4320 |
| 82% | Non-M | 100° C. | 7 | DDU-4320 |
| 85% | Non-M | 100° C. | 15 | DDU-4320 |
| 80% | Non-M | 100° C. | 30 | DDU-4320 |
| 75% | Non-M | 100° C. | 120 | DDU-4320 |
| 75% | M | 60° C. | 7 | DDU-4320 |
| 50% | M | 80° C. | 7 | DDU-4320 |
| 10% | M | 100° C. | 7 | DDU-4320 |
| 0% | M | 120° C. | 7 | DDU-4320 |
| 0% | M | 140° C. | 7 | DDU-4320 |
| 0% | M | 160° C. | 7 | DDU-4320 |
| 85% | Non-M | 60° C. | 7 | DDU-4320 |
| 83% | Non-M | 80° C. | 7 | DDU-4320 |
| 85% | Non-M | 100° C. | 7 | DDU-4320 |
| 83% | Non-M | 120° C. | 7 | DDU-4320 |
| 80% | Non-M | 140° C. | 7 | DDU-4320 |
| 58% | Non-M | 160° C. | 7 | DDU-4320 |

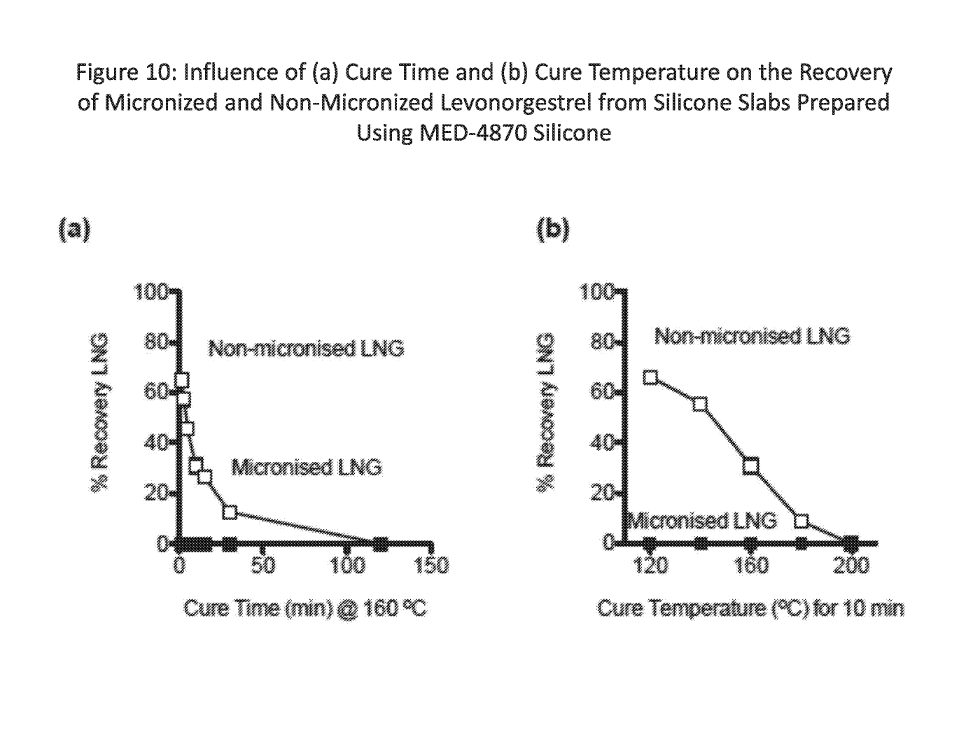

A similar experiment was conducted with the NuSil MED-4870 silicone elastomer system and the results are presented in FIGS. 10 (a) and (b). Higher cure temperatures were required for the NuSil MED-4870 system to ensure sufficient curing to allow for slab removal from the molds. As with the NuSil DDU-4320 silicone system, greater LNG recovery was observed with the non-micronized LNG compared with micronized LNG for all of the manufacturing conditions considered. In fact, with the higher cure temperatures and longer cure times required for NuSil MED-4870, no micronized LNG was recovered from any slab sample. In particular, at the temperature and time profile used in ring manufacture for the stability study (160° C. for 3 minutes), no LNG was recovered from slabs using micronized material. In contrast, non-micronized slabs prepared under the same cure conditions exhibited approximately 60% LNG recovery. These data correlate with recoveries obtained from rings prepared for the storage trial where recoveries of approximately 0.5% and 57% were obtained for rings made with micronized and non-micronized LNG respectively. The results of these experiments are depicted graphically, below.

| | Micronized | | | |
|---|---|---|---|---|
| | (M) or | | | |
| % LNG | Non-Micronized | Cure | Cure time | Type of |
| Recoverable | (Non-M) | temperature | (minutes) | silicone |
| 0% | M | 160° C. | 1.5 | MED-4870 |
| 0% | M | 160° C. | 3 | MED-4870 |
| 0% | M | 160° C. | 7 | MED-4870 |
| 0% | M | 160° C. | 15 | MED-4870 |
| 0% | M | 160° C. | 20 | MED-4870 |
| 0% | M | 160° C. | 30 | MED-4870 |
| 0% | M | 160° C. | 120 | MED-4870 |
| 65% | Non-M | 160° C. | 1.5 | MED-4870 |
| 58% | Non-M | 160° C. | 3 | MED-4870 |
| 47% | Non-M | 160° C. | 7 | MED-4870 |
| 33% | Non-M | 160° C. | 15 | MED-4870 |
| 28% | Non-M | 160° C. | 20 | MED-4870 |
| 10% | Non-M | 160° C. | 30 | MED-4870 |
| 0% | Non-M | 160° C. | 120 | MED-4870 |
| 0% | M | 120° C. | 10 | MED-4870 |
| 0% | M | 140° C. | 10 | MED-4870 |
| 0% | M | 160° C. | 10 | MED-4870 |
| 0% | M | 180° C. | 10 | MED-4870 |
| 0% | M | 200° C. | 10 | MED-4870 |
| 65% | Non-M | 120° C. | 10 | MED-4870 |
| 55% | Non-M | 140° C. | 10 | MED-4870 |
| 60% | Non-M | 160° C. | 10 | MED-4870 |
| 8% | Non-M | 180° C. | 10 | MED-4870 |
| 0% | Non-M | 200° C. | 10 | MED-4870 |

Similar to the data obtained for the NuSil DDU-4320 system, LNG recovery from the NuSil MED-4870 slabs was temperature and time dependent. However, unlike the NuSil DDU-4320 system, no plateaus were observed in either the temperature or time dependence of LNG loss. This difference between the two silicone systems may be due to different cure temperatures and times. However, from the perspective of LNG recovery, it is clear that a greater recovery is possible from the NuSil DDU-4320 compared with NuSil MED-4870 system, attributed to its lower cure temperature and time.

### Example 5: Optimization of Silicone Polymer Cure Conditions and Drug Particle Size

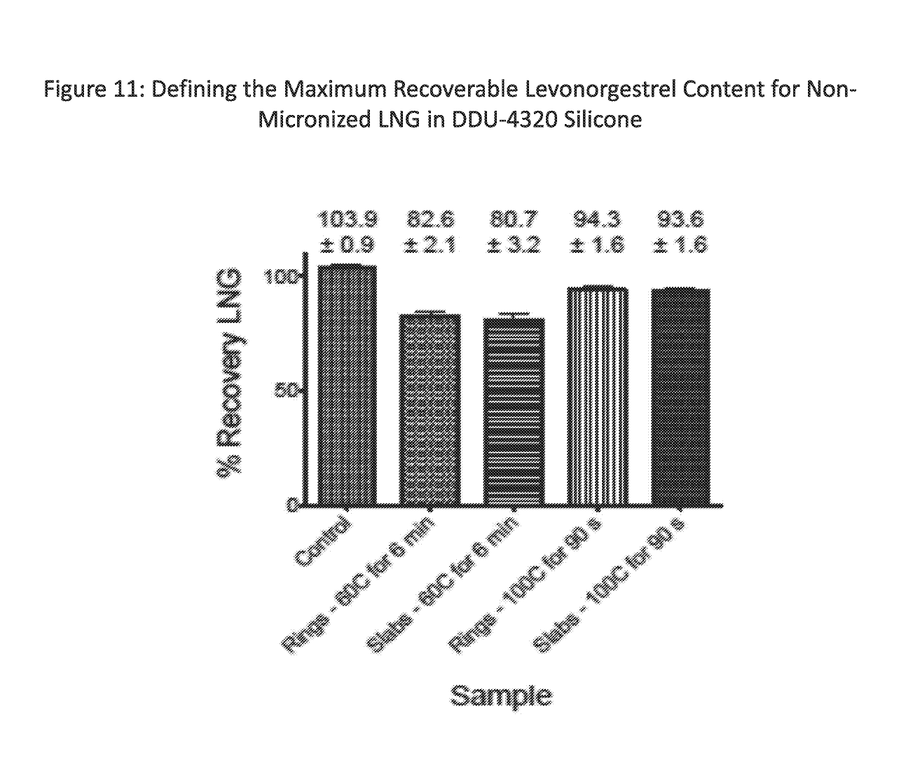

In an effort to define the maximum possible recovery of contraceptive using the best possible conditions for manufacture, further silicone slabs were prepared with non-micronized LNG. In this case, a low temperature manufacture at 60° C. with a cure time of 6 minutes was compared to a shorter higher temperature cure of 100° C. for 1.5 minutes. In addition, to define precisely the recovery of LNG expected from a ring compared to a slab, intravaginal rings were also prepared under the above conditions. The results are presented in FIG. 11, where the data shows that shorter cure time at 100° C. provided better recovery than the longer cure time at lower temperature.

In another experiment, silicone rings and slabs were prepared with non-micronized API having a terminal alkene, alkyne or carbonyl group (see Table, below). Cure temperatures ranging from 60° C. to 120° C. for 60 seconds to 360 seconds were studied.

| | | Cure time | | Type of | % API |
|---|---|---|---|---|---|
| D90* | Cure temp. (° C.) | (seconds) | Dimension of ring | silicone | Recoverable |
| 136 | 100° C. | 90 | 8 mm CSD, 54 mm OD | DDU-4320 | 95 |
| 80 | 100° C. | 90 | 8 mm CSD, 54 mm OD | DDU-4320 | 75 |
| 136 | 60° C. | 360 | 8 mm CSD, 54 mm OD | DDU-4320 | 83 |
| 136 | 60° C. | 360 | 5 cm × 3 cm × 0.5 cm | DDU-4320 | 81 |
| | | | “slab” | | |
| 140 | 120° C. | 600 | 5 cm × 3 cm × 0.5 cm | MED-4870 | 70 |
| | | | “slab” | | |
| 200 | 100° C. | 70 | 7 mm CSD, 52 mm OD | MED-4320 | 95 |
| 160 | 120° C. | 60 | 6 mm CSD, 50 mm OD | MED-4320 | 98 |

*D90 is the diameter at which 90% of the sample's measured particles are smaller sized particles.

### Example 6: Evidence of Compounds Binding to Silicone Elastomer at Both the Alkene, Alkyne or Carbonyl Groups

To determine the binding site of compounds to the silicone polymer, Raman spectroscopy studies were undertaken. In the first study, scanning Raman microscopy was used as a method for mapping drug distribution in silicone elastomer films. Thin films were created using NuSil DDU-4320 silicone elastomer and either dapivirine, micronized levonorgestrel, or non-micronized levonorgestrel. 400 points were sampled. In the second study, Raman microscopy was used as a tool to assess binding to poly(methylhydrosiloxane).

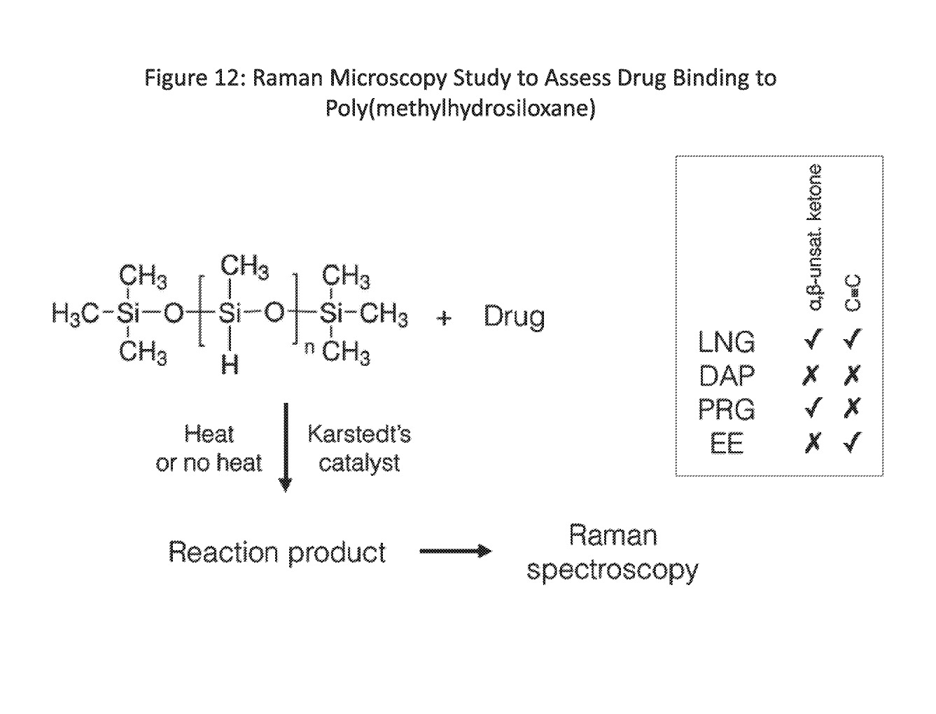

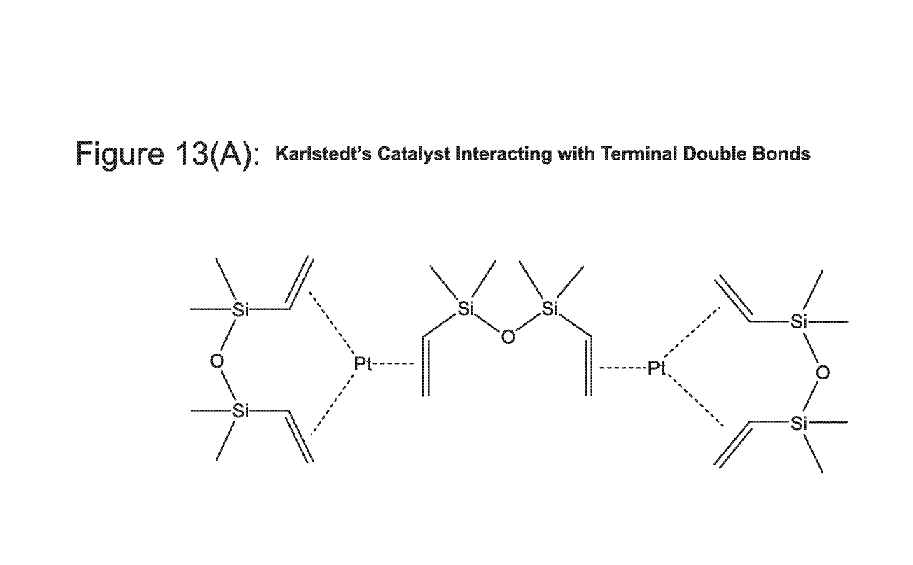

The results of these studies are depicted in FIG. 12, and confirm that compounds having terminal alkene or alkyne bonds, such as levonorgestrel (LNG) and ethylene estradiol (EE) covalently bind to the silicone elastomer via their terminal alkene or alkyne group. However, surprisingly, compounds lacking a terminal alkene or alkyne group but comprising a terminal carbonyl group, such as an α,β-unsaturated ketone, such as progesterone (PRG), also covalently bind to the silicone polymer. Compounds lacking either a terminal alkene, alkyne or carbonyl group, such as dapivirine (DAP) do not covalently bind to the silicone polymer. Thus, these studies provide evidence that compounds having either a terminal alkene or alkyne group, as well as compounds having a terminal carbonyl group, such as an α,β-unsaturated ketone group, covalently bind to the silicone elastomer.

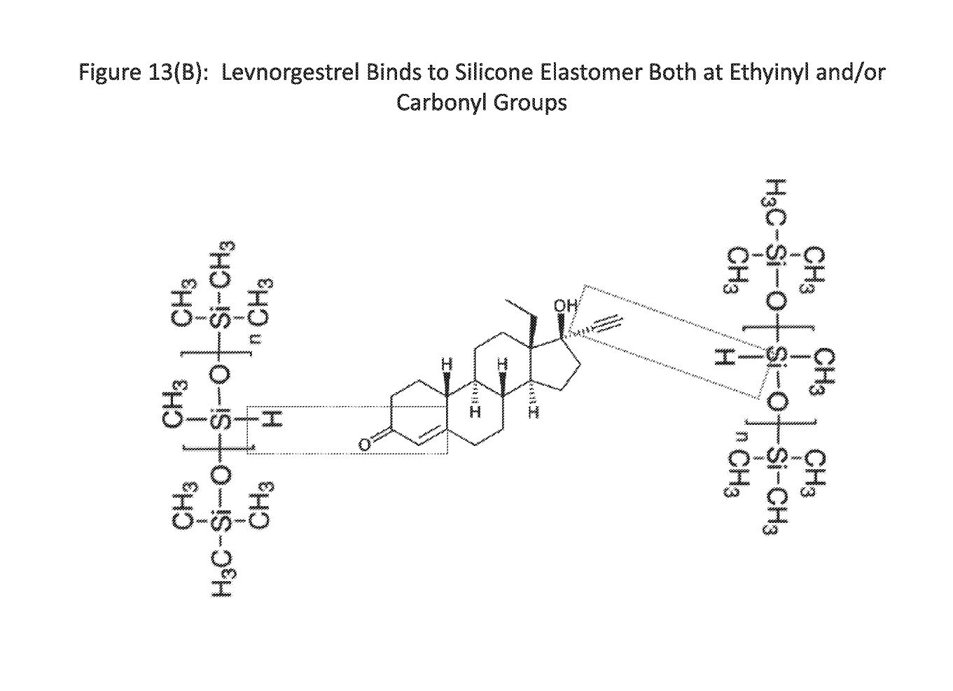

In summary, binding of compounds comprising a terminal alkene, alkyne or carbonyl group, such as levonorgestrel (LNG), to platinum-cured silicones is chemically favorable due to the triple bonds, double bonds, and carbonyl groups present on the molecule, interaction of these bonds with the platinum catalyst, and potential for Si—H groups binding to a terminal c-c double bond, triple bond, or carbonyl group, e.g., an α,β-unsaturated ketone. The cure chemistry of platinum cured silicone polymers involves a platinum catalyst (typically a Karlstedt's catalyst, see FIG. 13(A)), which interacts with the vinyl groups of the silicone base portion of a two-part addition cured silicone system. When compounds with terminal alkene or alkyne groups, or terminal carbonyl groups, are integrated into a platinum cure polymer system, these compounds may interfere with the catalyst (see FIG. 13(B)). This interaction is referred to as platinum catalyst poisoning. This presents a problem for the incorporation of drugs having available alkyne groups into addition-cure silicone elastomer drug delivery systems, such as intravaginal rings.

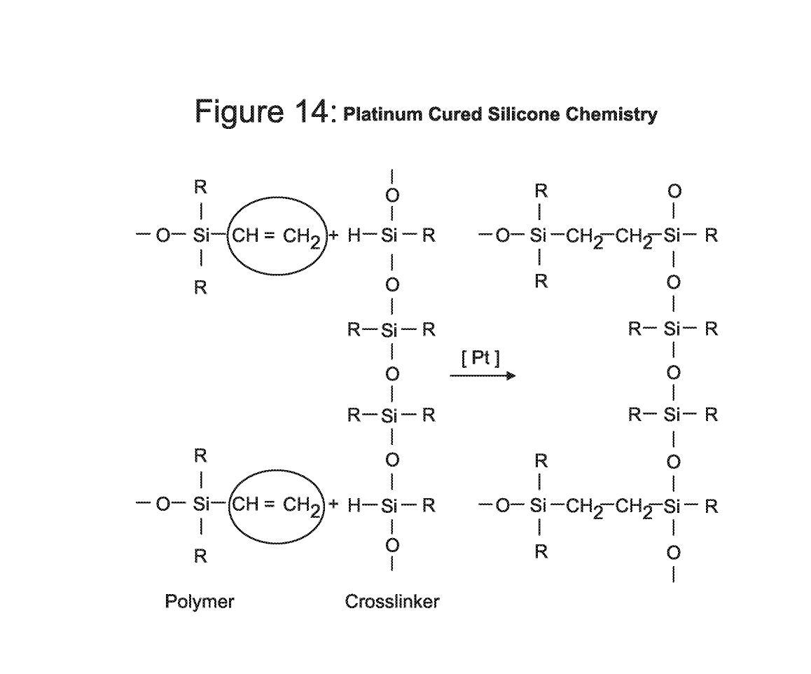

The results presented herein demonstrate that compounds having terminal alkenes, alkynes and/or carbonyl groups, such as levonorgestrel, interact with the platinum catalyst and substitute for the vinyl functional groups of the silicone backbone. This results in the compound becoming covalently and irreversibly bound to the silicone polymer. FIG. 14 shows the addition cured chemistry involved in two-part platinum cured silicones.

To date, no formulations have been discovered whereby compounds having a terminal alkene, alkyne and/or carbonyl group, have been incorporated into a platinum-cured silicone with drug recovery in the 75-100% range. The results presented herein demonstrate that the use of large compound particle size alone is not sufficient. If particles are large but primary particle size is small, then drug recovery is low (<50%). Moreover, the results presented herein demonstrate that use of optimal cure conditions, alone, is also insufficient. No cure conditions have been reached which result in high (50-100%) drug recovery. Only by minimizing the amount of compound having a terminal alkene, alkyne and/or carbonyl group solubilized in the silicone can the binding of the compound to silicone by minimized.

Thus, the instant invention provides a process by which the chemical binding of compounds having a terminal alkene, alkyne or carbonyl group to platinum cured silicone polymers is reduced to a range which is acceptable for drug delivery devices, such as intravaginal rings. The process by which binding of contraceptive to the silicone polymer is minimized must contain these two components:
1) active pharmaceutical ingredient with large primary particle size,
2) reduced thermal exposure of the formulation, particularly,
   a. use of a low-temperature cure silicone system, and/or
   b. optimized cure time.

Only by decreasing the amount of the compound having a terminal alkene, alkyne or carbonyl group solubilized in the silicone during the cure cycle will the amount of the compound bound to the silicone polymer be reduced to an acceptable level for commercial drug products, such as intravaginal rings. To again summarize, the amount of drug solubilized during the cure process is regulated by the rate of dissolution. Thus, the amount of drug which is solubilized after mixing into the silicone system is determined by three factors: 1) total surface area of the drug exposed to the silicone polymer; 2) the temperature of the system (higher temperature typically equals higher solubility of the drug in the system), and 3) the time the drug has been in contact with the silicone, as reaching equilibrium solubility is a time-dependent process.

Drug solubilization can be reduced by using highly crystalline material so that more energy is needed to solubilize the drug, by using larger crystal size which reduces the surface area to increase solubilization time, or reduce thermal exposure. Reducing thermal exposure can include a short cure time with high heat, a long cure time with low heat, cool starting materials, or by controlling the heat generated during mixing.

By optimizing platinum-catalyzed silicone drug delivery devices, such as intravaginal rings or IUDs, for drug particle size, cure temperature and cure time, the instant invention minimizes the amount of drug which binds to the silicone polymer during the platinum curing process, thereby resulting in higher release of the drug from the device.

### Example 7: Other Contraceptive Compounds of the Invention

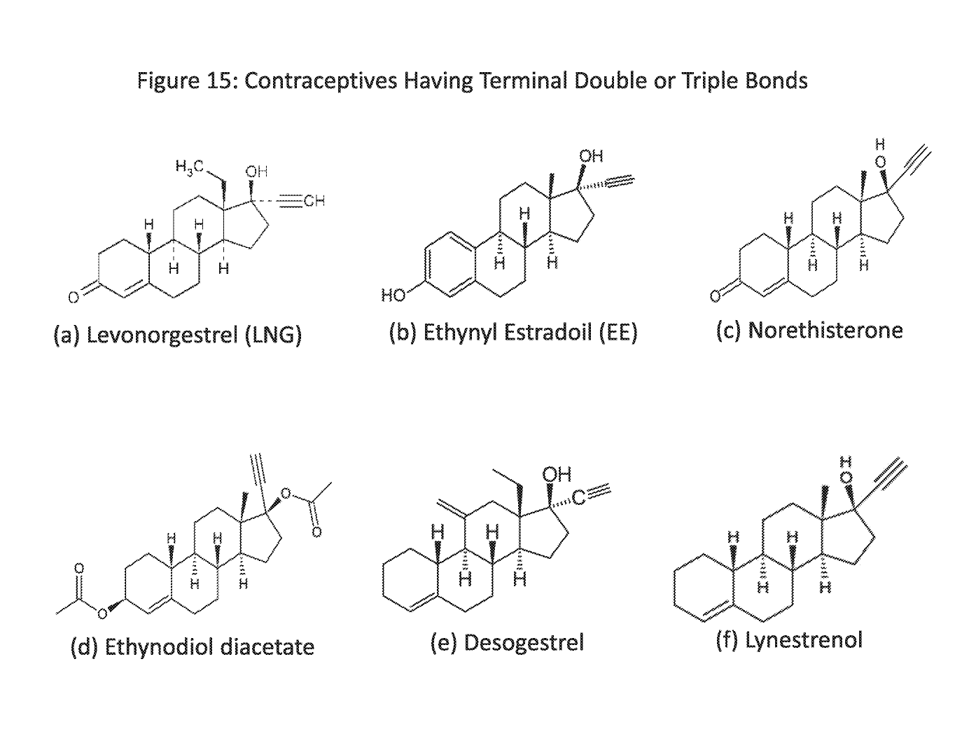

FIG. 15 depicts the chemical structure of levonorgestrel (LNG) and other contraceptive compounds in the same class: ethynyl estradiol (EE), norethisterone, ethynodiol diacetate, desogestrel, and lynestrenol. These compounds all contain a terminal alkyne, which has the potential to interact with the platinum catalyst and, thereby, bond at the silicon-hydride site on the silicone backbone.

For silicone slabs and intravaginal rings comprising multiple drugs, such as a contraceptive and dapivirine, the effect on the contraceptive is similar. However, since dapivirine is not chemically involved in the curing reaction, the presence of dapivirine is not necessary to prevent and does not affect the covalent binding of the contraceptive, such as levonorgestrel, to silicone polymers.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A platinum-catalyzed silicone intravaginal drug delivery device comprising a compound having a terminal alkene, a terminal alkyne, or a terminal carbonyl, wherein at least about 75% of the compound is recoverable from the device.

2. The intravaginal drug delivery device of claim 1, wherein at least about 80% of the compound is recoverable from the device.

3. The intravaginal drug delivery device of claim 1, wherein at least about 85% of the compound is recoverable from the device.

4. The intravaginal drug delivery device of claim 1, wherein at least about 90% of the compound is recoverable from the device.

5. The intravaginal drug delivery device of claim 1, wherein at least about 95% of the compound is recoverable from the device.

6. The intravaginal drug delivery device of claim 1, wherein at least about 99% of the compound is recoverable from the device.

7. A platinum-catalyzed silicone intravaginal drug delivery device comprising a compound having a terminal alkene, a terminal alkyne, or a terminal carbonyl, wherein about 100% of the compound is recoverable from the device.

8. A platinum-catalyzed silicone intravaginal drug delivery device comprising a compound having a terminal alkene, a terminal alkyne, or a terminal carbonyl, wherein less than about 25% of the compound is covalently bound to the silicone.

9. The intravaginal drug delivery device of claim 8, wherein less than about 20% of the compound is covalently bound to the silicone.

10. The intravaginal drug delivery device of claim 8, wherein less than about 15% of the compound is covalently bound to the silicone.

11. The intravaginal drug delivery device of claim 8, wherein less than about 10% of the compound is covalently bound to the silicone.

12. The intravaginal drug delivery device of claim 8, wherein less than about 5% of the compound is covalently bound to the silicone.

13. The intravaginal drug delivery device of claim 8, wherein less than about 2% of the compound is covalently bound to the silicone.

14. The intravaginal drug delivery device of claim 8, wherein less than about 1% of the compound is covalently bound to the silicone.

15. A platinum-catalyzed silicone intravaginal drug delivery device comprising a compound having a terminal alkene, a terminal alkyne, or a terminal carbonyl, wherein about 0% of the compound is covalently bound to the silicone.

16. The intravaginal drug delivery device of any one of claims 8-15, wherein the compound is covalently bound to a silicone hydride group on the silicone polymer.

17. The intravaginal drug delivery device of any one of the previous claims, wherein the compound is present in the device in a therapeutically effective amount.

18. The intravaginal drug delivery device of any one of the previous claims, wherein the compound is present in the device in a prophylactically effective amount.

19. The intravaginal drug delivery device of any one of the previous claims, wherein the compound has a large primary particle size.

20. The intravaginal drug delivery device of claim 19, wherein the compound has a d50 of about 40 microns to about 500 microns.

21. The intravaginal drug delivery device of claim 20, wherein the compound has a d50 of about 40 microns to about 250 microns.

22. The intravaginal drug delivery device of claim 21, wherein the compound has a d50 of about 55 microns to about 100 microns.

23. The intravaginal drug delivery device of claim 22, wherein the compound has a d50 of about 55 microns.

24. The intravaginal drug delivery device of claim 19, wherein the compound has a d90 of about 80 microns to about 500 microns.

25. The intravaginal drug delivery device of claim 24, wherein the compound has a d90 of about 80 microns to about 250 microns.

26. The intravaginal drug delivery device of claim 25, wherein the compound has a d90 of about 80 microns to about 150 microns.

27. The intravaginal drug delivery device of claim 26, wherein the compound has a d90 of about 135 microns.

28. The intravaginal drug delivery device of claim 19, wherein the compound has a primary particle size of about 40 microns to about 500 microns.

29. The intravaginal drug delivery device of any one of the previous claims, wherein the compound is non-micronized.

30. The intravaginal drug delivery device of any one of the previous claims, wherein the silicone is NuSil MED-4870 or NuSil DDU-4320.

31. The intravaginal drug delivery device of any one of the previous claims, wherein the compound is a contraceptive.

32. The intravaginal drug delivery device of claim 31, wherein the contraceptive is selected from the group consisting of levonorgestrel (LNG), ethynyl estradiol, noresthisterone, ethynodiol diacetate, desogestrel, and lynestrenol.

33. The intravaginal drug delivery device of claim 31, wherein the contraceptive is levonorgestrel.

34. The intravaginal drug delivery device of claim 31, wherein about 16 mg to about 64 mg of the contraceptive is present in the intravaginal drug delivery device.

35. The intravaginal drug delivery device of claim 34, wherein about 32 mg of the contraceptive is present in the intravaginal drug delivery device.

36. The intravaginal drug delivery device of any one of the previous claims, wherein the intravaginal drug delivery device further comprises an antimicrobial compound.

37. The intravaginal drug delivery device of claim 36, wherein the antimicrobial compound is dapivirine.

38. The intravaginal drug delivery device of claim 37, wherein about 25 mg to about 400 mg of dapivirine is present in the intravaginal drug delivery device.

39. The intravaginal drug delivery device of claim 38, wherein about 200 mg of dapivirine is present in the intravaginal drug delivery device.

40. The intravaginal drug delivery device of any one of the previous claims, wherein the intravaginal drug delivery device is a matrix-type intravaginal ring.

41. The intravaginal drug delivery device of any one of the previous claims, wherein the intravaginal drug delivery device is a reservoir-type intravaginal ring.

42. A method for producing a platinum-catalyzed silicone intravaginal drug delivery device comprising a compound having a terminal alkene, a terminal alkyne or a terminal carbonyl group, wherein at least 75% of the compound is recoverable from the device, the method comprising:
a) preparing a premix comprising the compound,
b) transferring the premix to an injection molder, and
c) curing the premix for about 60 seconds to about 10 minutes at a temperature of about 60° C. to about 120° C.,
wherein the compound has a primary particle size of about 40 microns to about 500 microns,
thereby producing the platinum-catalyzed silicone intravaginal drug delivery device.

43. The method of claim 42, wherein the premix is cured for about 60 seconds, about 90 seconds, about 3 minutes or about 10 minutes.

44. The method of claim 42, wherein the premix is cured at a temperature of about 60° C., about 80° C., about 100° C., or about 120° C.

45. The method of claim 42, wherein the compound has a d90 of about 80, about 90, about 100, about 110, about 120, about 130, about 135, about 140, about 160, about 180, or about 200.

46. A method of preventing pregnancy in a female human, comprising the step of inserting the intravaginal drug delivery device of any one of claims 1-39 into the vagina of the female human.

47. A method of preventing pregnancy and preventing HIV infection in a female human, comprising the step of inserting the intravaginal drug delivery device of any one of claims 1-39 into the vagina of the female human.
